(19) United States
(12) Patent Application Publication
(10) Pub. No.: US 2021/0340516 A1
(43) Pub. Date: Nov. 4, 2021

(54) COMPOSITIONS CONTAINING ARGINASE 1 FOR THE TREATMENT OF NEUROVASCULAR AND RETINAL VASCULAR DISORDERS

(71) Applicants: Augusta University Research Institute, Inc., Augusta, GA (US); The US Government as Represented by the Department of Veterans Affairs, Washington, DC (US)

(72) Inventor: Ruth CALDWELL, Augusta, GA (US)

(21) Appl. No.: 17/244,385

(22) Filed: Apr. 29, 2021

Related U.S. Application Data
(60) Provisional application No. 63/017,275, filed on Apr. 29, 2020.

(51) Int. Cl. C12N 9/78; A61K 47/60; A61P 27/02

(52) U.S. Cl. CPC C12N 9/78; A61K 47/60; A61K 38/00; C12Y 305/03001; A61P 27/02

(57) ABSTRACT

Pathological retinal neovascularization is a common micro-vascular complication in several retinal diseases including retinopathy of prematurity (ROP), diabetic retinopathy, age-related macular degeneration and central vein occlusion. Disclosed herein are compositions and methods useful for the treatment or prevention of retinal neovascularization and related diseases in a subject in need thereof. Exemplary methods include administering a composition including PEGylated arginase 1 to a subject in need thereof to promote reparative angiogenesis and decrease retinal neovascularization in the eye.

### CROSS REFERENCE TO RELATED APPLICATIONS

This application claims benefit of and priority to US Provisional Patent Application No. 63/017,275 filed on Apr. 29, 2020, and which is incorporated by reference in its entirety.

### STATEMENT REGARDING FEDERALLY SPONSORED RESEARCH

This invention was made with government support under EY011766 awarded by the National Institutes of Health. The government has certain rights in the invention.

### TECHNICAL FIELD OF THE INVENTION

This invention is generally related to compositions and methods of treating ischemic retinopathies, other retinal diseases, and acute central nervous systemic injuries.

### REFERENCE TO SEQUENCE LISTING

The instant application contains a Sequence Listing which has been submitted electronically in ASCII format and is hereby incorporated by reference in its entirety. Said ASCII copy, created on May 7, 2021, is named 064466_118_SL.txt and is 8,152 bytes in size.

### BACKGROUND OF THE INVENTION

Retinopathy of prematurity (ROP) is a potentially blinding eye disorder that affects premature infants born before the retinal vessels complete their normal development. Upon delivery, the neonate is immediately exposed to an environment that is relatively hyperoxic compared to the intra-uterine environment, and premature infants often require supplemental oxygen to support their immature respiratory system. Exposure to excessive oxygen levels is known to play a key role in ROP due to hyperoxia-induced damage of the immature vessels and/or inhibition of normal vascular development. As the neuronal development continues, the avascular areas become relatively hypoxic. This promotes the upregulation of vascular growth factors and can lead to pathological angiogenesis in which proliferating vessels infiltrate the vitreous. This may cause retinal detachment and blindness (Smith, L E, Investigative Ophthalmology & Visual Science, 49:5177-5182 (2008); Sapieha P., et al., The Journal of Clinical Investigation, 120:3022-3032 (2010)).

ROP is diagnosed on the basis of the retinal vascular pathology but is now recognized to be associated with long lasting neuronal damage (Moskowitz, A., et al., Eye and Brain, 8:103-111 (2016)). Current clinical therapies include cryotherapy, laser photocoagulation, and anti-vascular endothelial growth factor injections into the vitreous, all of which target pathological vitreoretinal neovascularization. However, in spite of effective control of the vascular pathology, many ROP patients still suffer lifelong vision loss, indicating that neuronal injury is a critical feature of the pathology. Thus, there is a great need for new therapies that promote physiological vascular repair and limit neuronal injury.

Many central nervous system (CNS) disorders involve a phase of ischemia or discontinuation of the blood supply. In the brain, this can manifest in the form of strokes and in the retina it can happen in different conditions that are collectively termed ischemic retinopathies. Examples of ischemic retinopathies include diabetic retinopathy, glaucoma, retinopathy of prematurity, and retinal artery or vein occlusion. Stroke is a leading cause of morbidity and mortality while ischemia-induced retinopathy is a leading cause of blindness and disability. While there are treatments and interventions for CNS ischemic conditions, they are often limited by the side effects, exclusion criteria or narrow time window making them not applicable for every patient. Therefore, there is a strong need for safe and effective treatments that can protect the CNS neurons against ischemia.

Therefore, it is an object of the invention to provide compositions and methods for the treatment or prevention of retinal neovascularization and neuronal injury.

It is another object of the invention to provide compositions and methods for the treatment of ROP, ischemic retinopathies, strokes and other diseases involving neuronal injury and vascular pathology.

### SUMMARY OF THE INVENTION

Pathological retinal neovascularization is a common micro-vascular complication in several retinal diseases including retinopathy of prematurity (ROP), diabetic retinopathy, age-related macular degeneration and central vein occlusion. Current therapeutic modalities of treating these diseases may slow or halt the progression of the disease but are unlikely to restore normal acuity. Disclosed herein are compositions and methods useful for the treatment or prevention of retinal neovascularization and related diseases in a subject in need thereof.

An exemplary method includes administering to the eye of the subject a composition including recombinant arginase 1 in an amount effective to promote reparative angiogenesis and decrease retinal neovascularization in the eye. In one embodiment, the recombinant arginase 1 is PEGylated. The recombinant arginase 1 can be administered to the eye of the subject by intravitreal injection or topical administration to the surface of the eye.

In some embodiments, the subjects with retinal neovascularization have retinopathy of prematurity (ROP), diabetic retinopathy, age-related macular degeneration, or retinal vein occlusion or are predisposed to develop any one of this pathologies.

In one embodiment, administration of the recombinant arginase 1 to the eye induces or promotes reparative angiogenesis in the eye by promoting an increase in the levels of growth factors in the retina. The growth factors can be FGF2 and CNTF. In another embodiment, the regression of retinal neovascularization is induced by decreasing the production and expression of inflammatory molecules in the retina. In some embodiments, the inflammatory molecules can be TNFα, IL-6, and MCP1.

In another embodiment, the systemic administration of recombinant arginase 1 induces a neuroprotective effect after ischemic stroke, retinal ischemia-reperfusion (IR) injury and traumatic optic neuropathy (TON).

Another embodiment provides method for treatment of ischemic stroke, retinal ischemia-reperfusion (IR) injury and traumatic optic neuropathy (TON) by systemic administration of recombinant arginase 1, wherein recombinant arginase 1 crosses the blood-brain and blood-retinal barriers after acute ischemic injury. The recombinant arginase 1 can be administered parentally, for example intravenously.

Another embodiment provides methods of treating CNS ischemic injuries administrating recombinant arginase 1 to a subject in need thereof. In some embodiment, in vivo expression of arginase 1 is induced. In some embodiments, arginase 1 expression is induced in specific treated tissues.

### BRIEF DESCRIPTION OF THE DRAWINGS

FIGS. 1A-1E are schematic illustrations of the experimental groups, treatments and sacrifice time points. WT and A1^{+/+} pups were subjected to oxygen-induced retinopathy (OIR) at post-natal day 7 (P7) and sacrificed during the vaso-obliteration phase (FIG. 1A), the neovascularization phase (FIG. 1B), or later at the age of twelve weeks (FIG. 1C). WT pups were treated with PEG-A1 or vehicle and sacrificed at the vaso-obliteration phase (FIG. 1D), or the neovascularization phase (FIG. 1E).

FIG. 2A is a representative Western blot image showing A1 expression at P13 in WT OIR retinas as compared to WT room air (RA) retinas. FIG. 2B is a graphical representation of the Western blotting quantification. *p<0.05 vs. RA group. N=3-4 mice. Data are presented as mean+−SD.

FIG. 3A is a graph showing arginase activity in WT pups and WT pups exposed to OIR. FIG. 3B is a graph showing A1 mRNA levels at P8 in WT mice and A2−/− mice. FIG. 3C is a representative Western blot image showing A1 and A2 protein expression in retinas from WT OIR mice and A2−/− OIR mice. FIG. 3D is a graph of A1 protein expression normalized to β-actin for WT and A2−/− OIR retinas.

FIGS. 4A and 4B are representative immunohistochemical (IHC) images showing lectin labelled retinal vessels from the retinas of WT and A1+/− mice that were placed in hyperoxia from P7 to P9. The dotted outline represents the area of capillary dropout. FIG. 4C is a graph showing quantification of the capillary dropout in WT and A1+/− mice. FIGS. 4D and 4E are representative IHC images showing lectin labelled retinal vessels from the retinas of WT and A1+/− mice that were placed in hyperoxia from P7 to P12. The dotted outline represents the area of capillary dropout. FIG. 4F is a graph showing quantification of the capillary dropout in WT and A1+/− mice. FIGS. 4G and 4H are representative IHC images showing lectin labelled retinal vessels from the retinas of WT and A1+/− mice that were subjected to OIR from P7 to P17. The dotted outline represents the area of capillary dropout. FIG. 4I is a graph showing quantification of the capillary dropout areas from the experiment of FIGS. 4G-4H. FIG. 4J is a graph showing quantification of Tufts areas from the experiment of FIGS. 4G-4H.

FIGS. 5A-5C are representative IHC images showing TUNEL labelling (FIGS. 5A-5B) and quantification (FIG. 5C) of the TUNEL labelled IHC images in retinas from WT OIR mice and A1+/− OIR mice. *p<0.05 vs WT OIR, N=6. FIGS. 5D-5H are representative IHC images showing calbindin labelling in RA WT (FIG. 5D), OIR WT (FIG. 5E), RA A1+/−(FIG. 5F), and OIR A1+/−(FIG. 5G), and quantification of the calbindin labelled IHC images (FIG. 5H). *p<0.05 vs WT RA, #p<0.05 vs. the other groups, N=4-6.

FIGS. 6A-6D are representative images of eye-globe cross-sections from OIR WT mice (FIGS. 6A-6B) and OIR A1+/− mice (FIGS. 6C-6D) labelled for calbindin and synaptophysin. Arrows refer to areas of horizontal cell loss.

FIGS. 7A-7D are representative images of retina cross-sections from RA WT mice (FIG. 7A), OIR WT mice (FIG. 7B), RA/A1+−/ (FIG. 7C), and OIR A1+/− mice (FIG. 7D) at P17 immunolabeled with GFAP. FIG. 7E is a representative Western blot image showing protein expression of GFAP in retinas from WT RA, WT OIR, A1+/− OIR, and A1+/−RA at P17. FIG. 7F is a graph showing quantification of the Western blots from FIG. 7E. FIGS. 7G-7J are representative images of SD-OCT analysis from 12 weeks old WT and A1+/− mice. FIGS. 7K-7M are graphs showing quantification of total retinal thickness (FIG. 7K), ganglion cell complex thickness (FIG. 7L), and ONL+IS thickness (FIG. 7M) from the SD-OCT analysis. FIGS. 7N-P are graphs showing quantification of total retinal thickness (FIG. 7N), ganglion cell complex thickness (FIG. 7O), and ONL+IS thickness (FIG. 7P) from SD-OCT analysis of the room air groups.

FIGS. 8A-8C are graphs showing visual acuity data for WT and A1+/− mice in room air or with OIR treatment for both eyes (FIG. 8A), left eye (FIG. 8B), and right eye (FIG. 8C).

FIG. 9 is a line graph showing arginase activity in the mouse vitreous after intravitreal PEG-A1 injection compared to baseline arginase activity.

FIG. 10A is a representative Western blot showing A1 protein level in OIR after intravitreal injection of PEG-A1. FIG. 10B is a graph showing the quantification of the Western blots from FIG. 10A.

FIGS. 11A-11B are representative IHC images showing lectin labelled retinal vessels from the retinas of WT mice treated with vehicle or PEG-A1 that were subjected to OIR. The dotted outline represents the area of capillary dropout. FIG. 11C is a graph showing quantification of the capillary dropout areas from the experiment of FIGS. 11A-11B. FIGS. 11D-11G are representative IHC images showing lectin staining and Iba-1 staining in retinas of WT mice treated with vehicle or PEG-A1 that were subjected to OIR. The arrows represent lectin-positive vitreal macrophages. FIG. 11H is a graph showing quantification of lectin-positive vitreal macrophages per field.

FIGS. 12A-12D are representative IHC images showing lectin labelled retinal vessels from the retinas of WT mice treated with vehicle or PEG-A1 that were subjected to OIR. The outline represents the area of capillary dropout. The arrowheads on FIGS. 12C and 12D indicate vascular tufts and vessel sprouts. FIGS. 12E-12G are graphs showing the quantification of tuft area (FIG. 12E), capillary dropout area (FIG. 12F), and vessel sprouts (FIG. 12G).

FIG. 13A is a representative Western blot showing cleaved Parp protein levels in WT mice treated with vehicle or PEG-A1 after exposure to room air (RA) or oxygen-induced retinopathy (OIR). FIG. 13B is a graph showing the quantification of the Western blots from FIG. 13A. FIGS. 13C-13F are representative IHC images showing calbindin labelled horizontal cells from retinas of WT mice treated with vehicle or PEG-A1 that were subjected to room air (RA) or oxygen-induced retinopathy (OIR). FIG. 13G is a graph showing quantification of calbindin-positive cells from the experiment of FIGS. 13C-13F.

FIGS. 14A-14H are graphs showing expression of retinal growth factors and inflammatory cytokines in retinas of WT mice treated with vehicle or PEG-A1 that were subjected to room air (RA) or oxygen-induced retinopathy (OIR) (FIG. 14A=TNF-α (tumor necrosis factor-α); FIG. 14B=IL-6 (interleukin-6); FIG. 14C=MCP1 (monocyte chemoattractant-1); FIG. 14D=iNOS (inducible nitric oxide synthase); FIG. 14E=A1 (arginase 1); FIG. 14F=FGF2 (fibroblast growth factor 2); FIG. 14G=CNTF (ciliary neurotrophic factor); FIG. 14H=IL-10 (interleukin 10)).

FIG. 15A is a representative Western blot showing FGF2 protein levels in the retinas of WT mice treated with vehicle or PEG-A1 after exposure to room air (RA) or oxygen-induced retinopathy (OIR). FIGS. 15B-15C are graphs showing the quantification of FGF2 monomer (FIG. 15B) and dimer (FIG. 15C) from the Western blots from FIG. 15A.

FIGS. 16A-16L show systemic PEG-A1 treatment is neuroprotective and crosses the blood-retina barrier after IR. FIG. 16A is a schematic representation of the experimental protocol for FIGS. 16B and 16C. FIGS. 16B and 16C are western blot images showing a comparison of vehicle to PEG-A1 treated mice perfused liver homogenates (16B) and perfused retinas collected at 24 hours after treatment of IR injured retinas compared to sham retinas from the same mice (16C). FIG. 16D is a schematic representation of the experimental protocol for FIGS. 16E-16FL. FIG. 16E-16G shows flat-mounts of retinas from PEG-A1 treated mice collected without perfusion at 7 days after treatment comparing immunofluorescent signals in the retina vessel network to the sham eye in the IR injured retinas. FIGS. 16H-16L show panels of NeuN staining (16H-16K) and quantification of retina flat-mounts (16L) collected at 7 days after retinal IR and PEG-A1 treatment as compared to vehicle treatment, n=6-8, *p<0.05 vs vehicle group.

FIGS. 17A-17G show systemic PEG-A1 treatment is neuroprotective and crosses the blood-retina barrier after stroke. FIG. 17A is a schematic representation of the experimental protocol for FIGS. 17B-17G. FIG. 17B is a western blot image of perfused brain homogenates comparing anti-PEG and A1 expression in middle cerebral artery occlusion (MCAO) compared to sham brains. FIG. 17C-17E shows representative triphenyltetrazolium chloride (TTC) red staining of the viable tissue in brain tissue sections highlights the unstained infarct area in white color in untreated (no ttt) and vehicle or PEG-A1 treated mice. FIG. 17F shows the quantification of the infarct size as a percent of contralesional hemisphere (*p<0.05 vs control). FIG. 17G shows the Bederson score of behavioral function after stroke. There was no difference in the infarct size or Bederson score between the untreated and the vehicle treated mice and therefore these groups were combined and identified by open and closed circles, respectively, n=611, *p<0.05 vs control group.

FIGS. 18A-18Q show systemic PEG-A1 treatment is neuroprotective and crosses the blood-retina barrier after ONC. FIG. 18A is a schematic representation of the experimental protocol for FIG. 18B. FIG. 18B is a western blot image of perfused retina and brain homogenates comparing anti-PEG expression in the retina/optic nerve (ON) subjected to ONC. FIG. 18C is a schematic representation of the experimental protocol for FIGS. 18D-18P. FIG. 18D-18L shows flat-mount immunolabeling using anti-PEG antibody and co-stained with lectin (stains vessels and some microglia/macrophages) showing PEG-A1 extravasation after ONC (white arrows). FIGS. 18M-18P and 18Q show flat-mount immunolabeling for the neuronal marker (NeuN) (18O) and quantification of PEG-A1 treated NeuN positive cells after ONC, n=4, *p<0.05 vs vehicle group.

FIGS. 19A-19K show PEG-A1 treatment protects retinal explants from oxygen-glucose deprivation/reoxygenation (OGD/R) injury ex vivo. FIG. 19A is a schematic representation of the experimental protocol for FIGS. 19B-19K. FIGS. 19B-19E and 19F are flat-mounts and bar graphs showing a time course for exposing retina explants to OGD/R ex vivo followed by fixation and flat-mount immunolabeling with NeuN, n=3 per group, *p<0.05 vs normoxia, **p<0.01 vs normoxia. FIGS. 19G-19J and 19K are flatmounts and bar graphs showing PEG-A1 treatment at reoxygenation significantly protected the retina explant neurons against OGD/R (3 hours/21 hours) as compared to control (OGD/R without treatment) or vehicle treated (PEG only) explants, n=8-10, *p<0.05 vs normoxia, ^{#}p<0.05 vs OGD/R control and vehicle groups.

FIGS. 20A-20E show PEG-A1 treatment does not provide neuroprotection to retinal neurons in vitro after OGD. FIG. 20A is a schematic representation of the experimental protocol for FIGS. 20B-20E. FIGS. 20B and 20C are bar graphs showing R28 retinal neurons subjected to OGD for 6 or 24 hours with or without PEG-A1 treatment (1 μg/mL) followed by LDH release assay on the supernatant. FIG. 20D is a bar graph of a MTT assay showing R28 cell viability after 3 hours of OGD and 3 hours of reoxygenation with a PEG-A1 control treatment, *p<0.05 vs control normoxia group. FIG. 20E shows primary rat retinal mixed neurons subjected to OGD/R (6 hours/18 hours) and treated at reperfusion with PEG-A1 (1 μg/mL) or ABH (100 μM). *p<0.05 vs control and PEG-A1 normoxia groups and ctrl OGD group, ^{#}p<0.05 vs control and ABH normoxia groups.

FIGS. 21A-21G show explants from control and myeloid A1 KO mice show no difference in neuronal cell death after OGD/R. FIG. 21A is a schematic representation of the experimental protocol for FIGS. 21B-21E and 21F. FIGS. 21B-21E and 21F show retina flatmount immunolabeling (21B-21E) and bar graphs (21F) after exposing retina explants from Ctrl and M-A1KO mice to OGD/R ex vivo., n=3-4, *p<0.05 vs normoxia. FIG. 21G is a western blot image of primary mouse macrophages treated with PEG-A1 for 18 hours suggesting.

FIG. 22A-22E are full uncropped western blot images wherein each image is labelled with a yellow box that refers to the representative band in the indicated figures. Note that the protein ladder used in all blots is Precision Plus Protein™ Dual Color Standards, cat #1610374, that shows marks for blue-stained bands (10, 15, 20, 37, 50, 100, 150, 250 kDa that are marked on the film as dots), in addition to 2 reference bands (25 and 75 kDa that are marked on the film with dashes).

FIG. 23A-23C show Z-stack retina flat-mount images which were processed using the ImageJ macro. The macro was validated for each set of images then applied to all images using ‘Batch analysis’ function of ImageJ. The macro creates a Z-project for each stack of images using maximum intensity projection. The macro then creates an automatic thresholding followed by ‘Analyze particles’ function with size exclusion below 25 μm to exclude non-specific speckles. If needed, user-defined exclusion of non-specific signals was performed manually. We found that signal area or integrated density is more reliable than particle count which can count a group of overlapping.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the terms “treat,” “treating,” “treatment” and “therapeutic use” refer to the elimination, reduction or amelioration of one or more symptoms of a disease or disorder. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of retinopathy. A therapeutically effective amount may also refer to the amount of the therapeutic agent that provides a therapeutic benefit in the treatment or management of a disease.

The terms “individual”, “host”, “subject”, and “patient” are used interchangeably herein, and refer to a mammal, including, but not limited to, humans, rodents, such as mice and rats, and other laboratory animals.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

An “immune cell” refers to any cell from the hemopoietic origin including, but not limited to, T cells, B cells, monocytes, dendritic cells, antigen presenting cells, and macrophages.

The term “inflammatory molecules” refer to molecules that result in inflammatory responses including, but not limited to, cytokines and metalloproteases such as including, but not limited to, IL-1β, TNF-α, TGF-beta, IFN-γ, IL-18, IL-17, IL-6, IL-23, IL-22, IL-21, and MMPs.

As used herein, “oxygen-induced retinopathy” or “OIR” refer to a mouse model of ischaemic vascular disease in the retina. The model is based on the exposure of mouse pups to hyperoxia during a phase when their retinal vasculature is still developing. This leads to capillary depletion, and upon return to room air, results in retinal ischaemia and proliferative vascular disease in the retinal vasculature.

As used herein, “retinopathy of prematurity” or “ROP” refer to an eye disease that primarily affects premature infants. ROP occurs when abnormal blood vessels grow and spread throughout the retina. Exposure to excessive oxygen levels through the administration of supplemental oxygen to the infant is known to play a key role in ROP due to hyperoxia-induced damage of the immature vessels and/or inhibition of normal vascular development.

As used herein, “neovascularization” refers to the natural formation of new blood vessels that form to serve as collateral circulation in response to local poor perfusion or ischemia. Neovascularization within the eye contributes to visual loss in several ocular diseases, including but not limited to proliferative diabetic retinopathy, neovascular age-related macular degeneration, and retinopathy of prematurity.

As used herein, “capillary dropout” refers to the closure of capillaries such that blood cannot travel through the capillary. Capillary dropout is a critical process in diabetic retinopathy, resulting in ischemia, release of angiogenic growth factors, and sight-threatening retinal neovascularization.

As used herein, “neovascular tufts” refer to biomicroscopic pathologic capillary outgrowths.

As used herein, “horizontal cells” refers to laterally interconnecting neurons having cell bodies in the inner nuclear layer of the retina of the eye. They help integrate and regulate the input from multiple photoreceptor cells.

As used herein, “hyperoxia” refers to a state in which oxygen supply is in excess.

### II. Compositions and Methods for Treating and Preventing Retinal Diseases and Neuronal Injuries

Pathological retinal neovascularization is a common micro-vascular complication in several retinal diseases including retinopathy of prematurity (ROP), diabetic retinopathy, age-related macular degeneration and central vein occlusion. In retinal neovascularization, new vessels invade the vitreous cavity, resulting in vision-threatening complications, such as vitreous hemorrhage and retinal detachment. The current therapeutic modalities of treating these diseases are invasive and although they may slow or halt the progression of the disease, they are unlikely to restore normal acuity. The lack of therapies to limit injury and promote repair during pathological retinal neovascularization is a major clinical problem. Therefore, there is a need to develop treatment modalities which are less invasive and therefore associated with fewer procedural complications and systemic side effects.

One embodiment provides a method of treating or preventing retinal neovascularization related ocular diseases in a subject in need thereof by increasing arginase 1 expression and activity in the eye. In one embodiment, arginase 1 expression and activity is increased in the eye by administering peglyated arginase 1 (PEG-A1). Arginase 1 can be administered by intravitreal administration or by topical administration to the eye.

Without being bound by any one theory, it is believed that PEG-A1 treatment decreases neovascularization and promotes angiogenic repair in retina with vascular disruption induced ischemic damage. The inflammatory molecules TNFα, IL-6 and MCP1 are commonly upregulated in the vitreous of patients with ischemic retinopathies (Demircan N, et al., Eye (London, England), 20:1366-1369 (2006); Mocan M C, et al., Canadian journal of ophthalmology, 41:747-752 (2006); Mitamura Y, et al. Japanese journal of ophthalmology, 46:218-221 (2002)), and have been shown to mediate pathological neovascularization in mouse models of OIR (Gardiner T A, et al., Am J Pathol, 166:637-644 (2005); Yoshida S, et al., Journal of leukocyte biology, 73:137-144 (2003)). In one embodiment, PEG-A1 treatment dampens or reduces the inflammatory response after ischemic retinopathy and decreases the expression of inflammatory molecules. The inducible enzyme, iNOS, plays an important role in pathological neovascularization and induces retinal thinning and cell apoptosis (Sennlaub F, et al., The Journal of clinical investigation, 107:717-725 (2001); Sennlaub F, et al., The Journal of neuroscience: the official journal of the Society for Neuroscience, 22:3987-3993 (2002)). In one embodiment, PEG-A1 treatment dampens, inhibits, or reduces the ischemic retinopathy-induced upregulation of iNOS.

In another embodiment, PEG-A1 treatment increases the levels of the growth factors in the retina. Retinal growth factors include but are not limited to fibroblast growth factors, insulin and insulin-like growth factors, transforming growth factor-beta, platelet-derived growth factors, nerve growth factors, epidermal growth factors and colony-stimulating factor. In one embodiment, PEG-A1 increases the levels of FGF2 and CNTF. Both FGF2 and CNTF have been shown to facilitate physiological neovascularization and reduce neovascular tuft formation (Bucher F, et al., Investigative ophthalmology & visual science, 57:4017-4026 (2016); Dorrell M I, et al., Glia, 58:43-54 (2010)). In one embodiment, PEG-A1 treatment improves ischemic retinopathy outcome by dampening, inhibiting, or reducing the retinal inflammatory response and upregulating production of neurotrophic factors.

One embodiment provides a method of treating or preventing retinal neovascularization induced injury and related diseases in a subject in need thereof by administering to the subject a composition including PEG-A1 in an amount effective to decrease neovascularization and to promote angiogenic repair in the retina. In one embodiment, PEG-A1 decreases the production and expression of inflammatory molecules in the retina. In another embodiment, PEG-A1 increases the production and expression of growth factors in the retina.

Intravitreal A1 treatment has been shown to improve neuronal survival in WT mice after retinal I/R injury. This intravitreal injection was administered using A1. The current disclosure obtained and used a pharmaceutical grade of PEG-A1 that is being tested in clinical trials. The mechanism of A1 protection may involve ameliorating the inflammatory response of infiltrating macrophages since myeloid specific A1 deletion using the LysM cre promoter, or global hemizygous deletion worsen the retinal IR-injury outcome (Fouda, A Y., et al., Cell Death & Disease, 9:1001 (2018)). Previous studies have shown LysM cre to be expressed mainly in macrophages, a subset of microglia and a small percentage of CNS neurons (Fouda, A Y, et al., Investigative Ophthalmology & Visual Science, 61:51 (2020); Orthgiess J, et al., European Journal of Immunology, 46:1529 (2016)).] In order to confirm that previous in vivo findings of worsened outcomes in mutant LysM cre-A1 floxed mice subjected to retinal IR injury are indeed due to myeloid cell-specific deletion of A1 and not due to its neuronal deletion, retina explants from control and LysM cre-A1 floxed mice were subjected to oxygen-glucose deprivation/reoxygenation injury. Unlike previous in vivo data that showed worsened outcome in the myeloid-specific A1 KO retinas, the ex vivo data showed no difference between the control and KO explants after OGD/R. This suggests that the in vivo phenotype seen in the previous study was indeed due to deletion of A1 in infiltrating myeloid cells and not off-target deletion in the neurons or to the loss of A1 in retinal microglia.

The role of arginase in acute CNS injury has been recently reviewed (Fouda, A Y, et al., Front Pharmacol., 11:277 (2020)). Both arginase isoforms have been shown to be expressed in the retina and brain tissues (Caldwell R W, et al., Physiological Reviews, 98:641 (2018)). A1 is widely used as a marker for M2-like reparative microglia/macrophages in CNS studies. Expression of A1 in these cells correlates with better stroke outcome (Hamzei, T S, et al., Biomaterials, 91:151 (2016)). Despite this, the direct action of A1 in stroke has not been studied to date. The disclosure herein presents the neuroprotective effect of A1 administration in stroke. A recent study showed that A1 is expressed by macrophages rather than microglia after permanent middle cerebral artery occlusion (Zarruk, J G, et al., Exp Neurol., 301:120 (2018)). Similarly, A1 has been shown to be upregulated in macrophages but not microglia after traumatic brain injury (TBI) (Hsieh, C L, et al., European journal of Immunology, 43:2010 (2013) One study showed improvement of stroke outcomes with the indirect arginase inhibitors, L-citrulline, L-ornithine and L-norvaline (Barakat, W, et al., Naunyn Schmiedebergs Arch Pharmacol., 391:603 (2018)). However, these amino acids have actions beyond arginase inhibition. This disclosure also presents neuroprotection with PEG-A1 treatment against traumatic optic neuropathy. This is in line with a previous report that showed neuroprotection against TBI with A1 overexpression in brain neurons (Madan, S, et al., Molecular Genetics and Metabolism, 125:112 (2018)).

The ex vivo retina explant model used herein is unique in providing the opportunity to test neuroprotective agents without involvement of systemic immune cells. The data show neuroprotection with PEG-A1 treatment ex vivo, which could be due to a direct protective effect on neurons or to an indirect effect on retinal glia. Interestingly, treatment with PEG-A1 in vitro did not show neuroprotection in two retina neuron cultures (primary mixed neurons and R28 cell line). A previous report showed a protective role of A1 overexpression in motor neurons against trophic factor deprivation in vitro (Estévez A G, et al., The Journal of Neuroscience, 26:8512 (2006)). However, the approach using PEG-A1 can lead to L-arginine depletion under in vitro conditions. L-arginine is a semi-essential amino acid in vivo, yet it is a required supplement in cell cultures (Morgan J F, et al., J Biol Chem., 233:664 (1958)). Furthermore, L-arginine supplementation has been shown to provide neuroprotection via preservation of arginase activity and formation of polyamines in motor neurons (Lee J, et al., Biochem Biophys Res Commun., 384:524 (2009)).

One embodiment provides methods for treating neurodegeneration in a subject in need thereof, comprising administering to the subject a composition comprising recombinant arginase 1 in an amount effective to promote neuroprotection or neurogenesis.

In one embodiment, neurodegeneration in a subject is the result of an ischemic injury to the brain or eye. In another embodiment neurodegeneration in a subject is the result of an acute retinal injury, resulting in traumatic optical neuropathy.

A. Methods for Increasing Arginase Activity in the Eye

Methods for increasing arginase expression and activity in the eye are provided herein. An exemplary method for increasing arginase expression and activity in the eye includes administering recombinant arginase 1 to the eye of a subject in need thereof. In one embodiment, the recombinant arginase 1 is PEGylated.

Arginase catalyzes the hydrolysis of arginine to ornithine and urea. At least two isoforms of mammalian arginase exist (arginase 1 (A1) and arginase 2 (A2)) which differ in their tissue distribution, subcellular localization, immunologic cross-reactivity and physiologic function (Ash, et al., Met Ions Biol Syst, 37:407-428 (2000)). A1, the cytosolic isoform, is strongly expressed in the liver, where it is a central player in the urea cycle (Morris, S. M. Jr., Annu Rev Nutr, 22: 87-105 (2002)). The mitochondrial isoform, A2, is expressed in extrahepatic tissues, especially the kidney (Miyanaka, K. et al. Histochem. J, 30:741-751 (1998)). Both isoforms are expressed in the retina and brain (Patel, C. et al. Front. Immunol., 4:173 (2013)), and have been linked to central nervous system (CNS) diseases (Caldwell, R. B., et al., Trends Pharmacol Sci, 36:395-405 (2015)). A1 is expressed in retinal glia. After experimental stroke, A1 has been reported to be strongest in myeloid cells with less expression in astrocytes (Hamzei Taj, S., et al., Biomaterials, 91:151-165 (2016); Quirie, A. et al. Eur. J Neurosci, 37:1193-1202 (2013)). A1 and nitric oxide synthase (NOS) enzyme compete for their common substrate the semi-essential amino acid L-arginine (Wu, G. & Morris, S. M. Jr., Biochem J., 336(Pt 1): 1-17 (1998)). A1 upregulation can lead to suppression of nitric oxide (NO) formation by endothelial NOS (eNOS) resulting in superoxide production, endothelial dysfunction, platelet aggregation, and leukocyte activation and attachment to the vessel wall (Caldwell, R. W., et al., Physiol. Rev., 98: 641-665 (2018)). However, A1 expression in “M2-like” anti-inflammatory myeloid cells is thought to reduce NO production by iNOS, and thus can dampen oxidative stress and inflammation (Munder, M. et al. J. Immunol. 163:3771-3777 (1999); Rath, M., et al. Front. Immunol. 5:532 (2014)). Interestingly, the number of A1+, Iba1+ macrophages/microglia is correlated with post-stroke neuron survival and recovery in mice11. Recent studies have shown that A1 is expressed exclusively by infiltrating myeloid cells and not by microglia after CNS injury (Greenhalgh, A. D. et al. Brain Behav. Immun. 56: 61-67 (2016); Zarruk, J. G., et al. Exp. Neurol. 301 (Pt. B): 120-132 (2018)).

While A2 plays a deleterious role in retinal ischemia, the data presented herein shows that A1 is protective (Fouda, A Y, et al., Cell death & Disease, 9:1001 (2018); Shosha E, et al., Cell death & disease, 7:e2483-e (2016)). Recent studies using a mouse model of acute retinal ischemia have shown worsened outcomes in mice lacking A1. Furthermore, intravitreal injection of A1 showed neuroprotection in this model (Fouda, A Y, et al., Cell death & Disease, 9:1001 (2018)).

Sequences for arginase 1 are known in the art. The amino acid sequence for arginase 1 isoform 1 is as follows:

```
                10 20 30 40
MSAKSRTIGI IGAPFSKGQP RGGVEEGPTV LRKAGLLEKL
                50 60 70 80
KEQECDVKDY GDLPFADIPN DSPFQIVKNP RSVGKASEQL
                90 100 110 120
AGKVAEVKKN GRISLVLGGD HSLAIGSISG HARVHPDLGV
              130 140 150 160
IWVDAHTDIN TPLTTISGNL HGQPVSFLLK ELKGKIPDVP
              170 180 190 200
GFSWVTPCIS AKDIVYIGLR DVDPGEHYIL KTLGIKYFSM
              210 220 230 240
TEVDRLGIGK VMEETLSYLL GRKKRPIHLS FDVDGLDPSF
              250 260 270 280
TPATGTPVVG GLTYREGLYI TEEIYKTGLL SGLDIMEVNP
              290 300 310 320
SLGKTPEEVT RTVNTAVAIT LACFGLAREG NHKPIDYLNP PK
(SEQ ID NO: 1, UniProt Accession P05089-1 which is incorporated by reference in its entirety).
```

One embodiment provides a recombinant arginase 1 protein having at least 50%, 60%, 70%, 80%, 85%, 90%, 95%, 97%, 98%, 99%, or 100% sequence identity to SEQ ID NO:1.

The amino acid sequence for arginase 1 isoform 2 (also known as erythroid variant) is as follows:

```
                10 20 30 40
MSAKSRTIGI IGAPFSKGQP RGGVEEGPTV LRKAGLLEKL
                50 60 70 80
KEQVTQNFLI LECDVKDYGD LPFADIPNDS PFQIVKNPRS
                90 100 110 120
VGKASEQLAG KVAEVKKNGR ISLVLGGDHS LAIGSISGHA
              130 140 150 160
RVHPDLGVIW VDAHTDINTP LTTTSGNLHG QPVSFLLKEL
              170 180 190 200
KGKIPDVPGF SWVTPCISAK DIVYIGLRDV DPGEHYILKT
              210 220 230 240
LGIKYFSMTE VDRLGIGKVM EETLSYLLGR KKRPIHLSFD
              250 260 270 280
VDGLDPSFTP ATGTPVVGGL TYREGLYITE EIYKTGLLSG
              290 300 310 320
LDIMEVNPSL GKTPEEVTRT VNTAVAITLA CFGLAREGNH
              330
KPIDYLNPPK
(SEQ ID NO: 2, UniProt accession P05089-2 which is incorporated by reference in its entirety).
```

One embodiment provides a recombinant arginase 1 protein having at least 50%, 60%, 70%, 80%, 85%, 90%, 95%, 97%, 98%, 99%, or 100% sequence identity to SEQ ID NO:2.

The amino acid sequence for arginase 1 isoform 3 is as follows:

```
                10 20 30 40
MSAKSRTIGI IGAPFSKGQP RGGVEEGPTV LRKAGLLEKL
                50 60 70 80
KEQECDVKDY GDLPFADIPN DSPFQIVKNP RSVGKASEQL
                90 100 110 120
AGKVAEVKKN GRISLVLGGD HSLAIGSISG HARVHPDLGV
              130 140 150 160
IWVDAHTDIN TPLTTTSGNL HGQPVSFLLK ELKGKIPDVP
              170 180 190 200
GFSWVTPCIS AKDIVYIGLR DVDPGEHYIL KTLGIKYFSM
              210 220 230
TEVTRTVNTA VAITLACFGL AREGNHKPID YLNPPK
(SEQ ID NO: 3, UniProt Accession P05089-3 which is incorporated by reference in its entirety).
```

One embodiment provides a recombinant arginase 1 protein having at least 50%, 60%, 70%, 80%, 85%, 90%, 95%, 97%, 98%, 99%, or 100% sequence identity to SEQ ID NO:3.

1. PEG-A1

In one embodiment, the A1 protein is PEGylated. Pegylation is a recognized way of preserving protein integrity and reducing immune reactions. PEG-BH-100, a PEGylated-A1 protein, is an investigational drug that is currently being tested in clinical trials for certain types of cancers that are dependent on arginine. PEG-BH-100 is a recombinant human arginase I covalently attached, via a succinamide propionic acid (SPA) linker, to a polyethylene glycol (PEG) of molecular weight 5,000. PEG-BH-100 has been shown to be safe and well-tolerated in humans.

Lipophilic drugs of molecular weight less than 500 Da can readily cross the blood-brain and blood-retina barriers (Pardridge, W M., et al., J Cereb Blood Flow Metab., 32:1959 (2012); Del Amo, E M, et al., Progress in Retinal and Eye Research. 57:134-85 (2017)). PEG-A1 is a hydrophilic drug. The recombinant A1 (37 kDa) enzyme is linked to multiple PEG (5 kDa) molecules and migrates on SDS-PAGE as a 220 kDa band. PEG-A1 was previously reported to exhibit poor CNS penetration (De Santo, C, et al., International Journal of Cancer, 142:1490-502 (2018)). The present disclosure shows limited PEG-A1 penetration into the sham retinas or brains after i.p. administration while the penetration is greatly enhanced with IR-injury or stroke and breakdown of the blood-retina and blood-brain barriers. In one embodiment PEG-A1 is systemically administered in acute and chronic CNS ischemic injury conditions where the blood-brain or the blood-retina barriers are compromised or disrupted. Furthermore, data presented herein shows PEG-A1 uptake by macrophages in vitro.

In one embodiment, recombinant arginase 1 is covalently attached to a PEG molecule. PEG can have a molecular weight from about 550 daltons to about 10,000 daltons. In another embodiment, the molecular weight of PEG is 600 daltons, 700 daltons, 800 daltons, 900 daltons, 1,000 daltons, 2,000 daltons, 3,000 daltons, 4,000 daltons, 5,000 daltons, 6,000 daltons, 7,000 daltons, 8,000 daltons, 9,000 daltons, or 10,000 daltons. In another embodiment, the molecular weight of PEG is 5,000 daltons.

One embodiment provides a method of treating retinal neovascularization and related diseases such as retinopathy of prematurity (ROP), diabetic retinopathy, age-related macular degeneration and central vein occlusion, by topically or intravitreally administering to an eye of the subject an effective amount of PEG-A1.

Upon administration into the eye, PEG-A1 metabolizes the amino acid arginine to ornithine and urea, depleting intraocular arginine. Without being bound by any one theory, it is believed that the depletion of arginine dampens, reduces, or inhibits the inflammatory response induced by retinopathy and decreases the expression of inflammatory molecules.

B. Pharmaceutical Compositions

Pharmaceutical compositions including PEG-A1 are provided. Pharmaceutical compositions containing the compounds can be for administration by parenteral (intramuscular, intraperitoneal, intravenous (IV) or subcutaneous injection, intravitreal) routes of administration, topical administration, or using bioerodible inserts and can be formulated in dosage forms appropriate for each route of administration. In one embodiment the formulation is an ophthalmic formulation. The ophthalmic formulation can be eye drops, ophthalmic solutions, microemulsions, sol-to-gel systems, ointments, ocular inserts (including but not limited to small oval wafers, produced from acrylamide, N-vinylpyrrolidone, and ethyl acrylate), nanoparticles, microparticles, or a combination thereof.

In certain embodiments, the PEG-A1 composition is administered locally, for example by injection directly into the eye. The PEG-A1 compositions can be combined with a matrix to assist in creating an increased localized concentration of the compositions by reducing the passive diffusion of the PEG-A1 out of the site to be treated.

In other embodiments, the PEG-A1 composition is administered systemically in acute and chronic CNS ischemic injury conditions where the blood-brain or the blood-retina barriers are compromised or disrupted.

In some embodiments, the amount of PEG-A1 in the formulation can be from 1.0 mg/mL to 100 mg/mL, about 1.0 mg/mL to about 50 mg/mL protein, about 5.0 mg/mL to about 25 mg/mL. In another embodiment, the amount of PEG-A1 in the formulation can be about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL. The composition can be formulated to achieve an intravitreal concentration of about 0.5 μg/mL.

1. Formulations for Parenteral Administration

In some embodiments, the disclosed PEG-A1 compositions are administered in an aqueous solution, by parenteral injection, typically by intravitreal or topical administration. The formulation may also be in the form of a suspension or emulsion. In general, pharmaceutical compositions are provided including effective amounts of PEG-A1, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

In some embodiments, the PEG-A1 compound is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before all of the first agent is released. In still other embodiments, both agents are released at the same time over the same period of time or over different periods of time.

In one embodiment, the extended release composition includes microparticles having a diameter from about 1 μm to about 10 μm. The microparticles can have a diameter of 1 μm, 2 μm, 3 μm, 4 μm, 5 μm, 6 μm, 7 μm, 8 μm, 9 μm, or 10 μm. In another embodiment, the extended release compositions include nanoparticles have a diameter ranging from 10 nm to 950 nm. The nanoparticles can have a diameter of 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 950 nm.

In some embodiments, the PEG-A1 composition releases the PEG-A1 at a steady rate for 30, 60, 90, or 120 days after administration.

2. Controlled Delivery Polymeric Matrices

The compositions disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of the disclosed compositions, although in some embodiments biodegradable matrices are preferred. These may be natural or synthetic polymers, although synthetic polymers are preferred in some embodiments due to the better characterization of degradation and release profiles. The polymer is selected based on the period over which release is desired. In some cases, linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the eye.

3. Topical Administration

Topical dosage forms of the disclosed PEG-A1 compositions include, but are not limited to eye drops, eye ointments, solutions, creams, emulsions, and other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). In one embodiment, the disclosed PEG-A1 compositions are delivered to the eye in a suitable topical dosage form. Topical dosage forms can include additional components that improve the efficiency of the administration of the PEG-A1 to the retina. In one embodiment, increasing the formulation's viscosity extends the time period of contact with the cornea and improves bioavailability of the active composition. Substances which can increase the viscosity include hydrophilic polymers of high molecular weight which do not diffuse through biological membranes and which form three-dimensional networks in the water. Examples of such polymers include but are not limited to polyvinyl alcohol, poloxamers, hyaluronic acid, carbomers, and polysaccharides, that is, cellulose derivatives, gellan gum, and xanthan gum. In another embodiment, penetration increasing substances are included in the topical formulation. Exemplary substances that increase penetration of topical formulations include but are not limited to chelating agents, preservatives, surfactants, and bile acid salts.

C. Dosing Regimen

In some in vivo approaches, the PEG-A1 compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed PEG-A1 compositions, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed PEG-A1 compositions, generally dosage levels of 0.001 to 20 mg/kg of body weight daily are administered to mammals.

In one embodiment, a pharmaceutical composition including PEG-A1 is administered into the eye of a subject in need thereof. In such an embodiment, the composition can be administered by intravitreal injection or by application of a topical composition to the surface of the eye. In a preferred embodiment, the composition is intravitreally injected into the eye. In one embodiment, the composition is administered to the subject once a week. In another embodiment, the composition is administered to the subject every other week, once a month, once every two months, once every three months, once every four months, once every five months, or once every six months. In some embodiments, the subject receives injections once a month for the first five injections, and then once every two months for the remainder of the treatment. In some embodiments, the subject receives the injections until the symptoms of their disease are reduced and then receive less frequent maintenance doses as needed. In other embodiments, the subject receives the injections on an as needed basis, for example as symptoms begin to reappear.

In the case of the topical administration route, the composition can be administered more frequently since it is not an invasive method. In such an embodiment, the topical composition can be applied once daily, twice daily, or three times daily. In another embodiment, the topical composition is administered every other day, twice per week, or once per week.

Suitable routes of administration of the PEG-A1 composition include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred. For example, for instances in which the compound is not orally bioavailable, intravenous injection may be a preferred route of administration.

One embodiment provides a method administering PEG-A1 compositions provided by intravenous injection. In other embodiments, the pharmaceutical compositions provided are administered intrathecally, endovascularly or intranasally.

In another embodiment PEG-A1 compositions may be combined with the administration of one or more other therapeutic agents. In particular, PEG-A1 may be combined with the administration of one or more therapeutic agents that may be effective in the treatment or prevention of stroke or damage resulting from stroke onset or of ischemic disorders or ischemic damage. Such agents include thrombolytics (e.g., streptokinase, tPA, antistreplase, reteplase, urokinase and tenecteplase); fibrinolytics (Retavase®); antiplatelet agents or platelet antiaggregants (e.g., aspirin, ticlopidine, clopidogrel and dipyridamole); anticoagulants (e.g., warfarin, heparin, ximelagatran); antihypertensives, including diuretics such as bumetamide, chlorothiazide, chlorthalidone, furosemide, hydrochlorothiazide, metolazone, and spironolactone; statins (e.g., HMG-CoA reductase inhibitors); beta blockers (e.g., atenolol, bisoprolol, carvedilol, metoprolol, and propranolol); ACE inhibitors (e.g., benazepril, captopril, enalapril, fosinopril, lisinopril, moexipril, quinapril, ramipril, and trandolapril); angiotensin II receptor antagonists (e.g., candesartan, irbesartan, losartan, potassium, and valsartan); calcium channel blockers (e.g., amlodipine, bepridil, diltiazem, felodipine, isradipine, nicardipine, nimodipine, nisoldipine, and verapamil); angiotensin receptor blockers (ARBs); thrombin inhibitors (e.g., warfarin, Ximelagatran); and other drugs that can lower blood pressure (e.g., clonidine, guanfacine, hydralazine, methyldopa, minoxidil, and prazosin); and neuroprotectants (e.g., NXY-059). Such agents may be administered in simultaneous, separate, or sequential (i.e., before or after) administration with the agents presented herein.

A combination therapy regimen may be additive, or it may produce synergistic results (e.g., a reduction of ischemic injury greater than expected for the combined use of the two agents). The compositions can be administered simultaneously or sequentially by the same or different routes of administration. The determination of the identity and amount of the pharmaceutical compositions for use in the methods presented herein can be readily made by ordinarily skilled medical practitioners using standard techniques known in the art.

D. Subjects to be Treated

Retinal neovascularization induced retinal injury is a primary contributor in blinding diseases such as retinopathy of prematurity (ROP), diabetic retinopathy, age-related macular degeneration and retinal vein occlusion. The disclosed methods and compositions can be used to treat or reduce the symptoms of diseases that result from retinal neovascularization-induced retinal injury. Subjects to be treated include humans, human infants, or human newborn.

1. Retinopathy of Prematurity

In one embodiment, the disclosed methods and compositions can be used to treat or prevent the progression of retinopathy of prematurity (ROP). ROP is an eye disease that primarily affects premature infants. ROP primarily affects premature infants weighing about 2¾ pounds (1250 grams) or less that are born before 31 weeks of gestation. The smaller a baby is at birth, the more likely that baby is to develop ROP. The disorder typically develops in both eyes and is one of the most common causes of visual loss in childhood and can lead to lifelong vision impairment and blindness. In addition to birth weight and how early a baby is born, other factors contributing to the risk of ROP include anemia, blood transfusions, respiratory distress, breathing difficulties, and the overall health of the infant.

ROP occurs when abnormal blood vessels grow and spread throughout the retina. Exposure to excessive oxygen levels through the administration of supplemental oxygen to the infant is known to play a key role in ROP due to hyperoxia-induced damage of the immature vessels and/or inhibition of normal vascular development. These abnormal blood vessels are fragile and can leak, scarring the retina and pulling it out of position. This causes a retinal detachment. Retinal detachment is the main cause of visual impairment and blindness in ROP.

ROP can occur in varying stages of severity which will influence the type and duration of treatment that the infant received. Stage I is characterized by mildly abnormal blood vessel growth. Many children who develop stage I improve with no treatment and eventually develop normal vision. The disease resolves on its own without further progression. Stage II is characterized by moderately abnormal blood vessel growth. Many children who develop stage II improve with no treatment and eventually develop normal vision. The disease resolves on its own without further progression. Stage III is characterized by severely abnormal blood vessel growth. The abnormal blood vessels grow toward the center of the eye instead of following their normal growth pattern along the surface of the retina. Some infants who develop stage III improve with no treatment and eventually develop normal vision. However, when infants have a certain degree of Stage III and “plus disease” develops, treatment is considered. “Plus disease” means that the blood vessels of the retina have become enlarged and twisted, indicating a worsening of the disease. Treatment at this point has a good chance of preventing retinal detachment. Stage IV is characterized by partially detached retina. Traction from the scar produced by bleeding, abnormal vessels pulls the retina away from the wall of the eye. Stage V is characterized by completely detached retina and the end stage of the disease. If the eye is left alone at this stage, the baby can have severe visual impairment and even blindness.

One embodiment provides a method of treating, reducing, or inhibiting the symptoms of ROP or reducing or inhibiting the progression of ROP in a subject in need thereof by administering to the subject a composition including PEG-A1 in an amount effective to decrease neovascularization and promote angiogenic repair in the retina. In one embodiment, PEG-A1 decreases the expression of inflammatory molecules in the eye. In another embodiment, PEG-A1 increases the production and expression of growth factors in the retina.

In one embodiment, a pharmaceutical composition including PEG-A1 is administered to premature infants who have been diagnosed with ROP. In one embodiment, the infants have Stage III, Stage IV, or Stage V ROP. In such embodiments, PEG-A1 is intravitreally injected into the affected eye or eyes in an amount effective to decrease neovascularization and promote angiogenic repair. Administration of PEG-A1 can halt progression of the disease and reverse disease related damage in the retina.

2. Diabetic Retinopathy

In one embodiment, the disclosed methods and compositions can treat the symptoms of or prevent the progression of diabetic retinopathy. Diabetic retinopathy is a diabetes related complication that affects the vasculature in the eye. Over time, too much sugar in the blood can lead to the blockage of blood vessels that nourish the retina, cutting off its blood supply. As a result, the eye attempts to grow new blood vessels. But these new blood vessels don't develop properly and can leak easily. The condition can develop in anyone who has type 1 or type 2 diabetes. The longer a subject has diabetes and the less controlled their blood sugar is, the more likely the subject is to develop diabetic retinopathy.

Early diabetic retinopathy, also called nonproliferative diabetic retinopathy (NPDR), is the more common form of diabetic retinopathy. In this type of retinopathy, new blood vessels are not proliferating. The walls of the blood vessels in the retina weaken and tiny bulges called microaneurysms protrude from the vessel walls of the smaller vessels and leak fluid and blood into the retina. In addition, larger retinal vessels can begin to dilate and become irregular in diameter. Early diabetic retinopathy can progress from mild to severe, as more blood vessels become blocked. Nerve fibers in the retina may begin to swell. Sometimes the central part of the retina (macula) begins to swell (macular edema), a condition that requires treatment.

Advanced diabetic retinopathy is the more severe type of retinopathy, also known as proliferative diabetic retinopathy. In this type of diabetic retinopathy, damaged blood vessels close off, causing the growth of new, abnormal blood vessels in the retina which can leak into the vitreous of the eye. Scar tissue stimulated by the growth of new blood vessels can cause the retina to detach from the back of the eye. If the new blood vessels interfere with the normal flow of fluid out of the eye, pressure can build up in the eye. This can lead to damage to the optic nerve, resulting in glaucoma.

One embodiment provides a method of treating the symptoms of or preventing the progression of diabetic retinopathy in a subject in need thereof by administering to the subject a composition including PEG-A1 in an amount effective to decrease neovascularization and promote angiogenic repair in the retina. In one embodiment, the subject has early diabetic retinopathy. In another embodiment, the subject has advanced diabetic retinopathy. In such embodiments, PEG-A1 reduces further disease related damage to the retina. In one embodiment, PEG-A1 decreases the expression of inflammatory molecules in the eye. In another embodiment, PEG-A1 increases the production and expression of growth factors in the retina.

In another embodiment, PEG-A1 is administered to the subject prophylactically, especially when the subject's blood sugar is not under control, to prevent the induction of diabetic retinopathy.

3. Branch Retinal Vein Occlusion

In one embodiment, the disclosed methods and compositions can treat the symptoms of or prevent the progression of branch retinal vein occlusion (BRVO). Retinal vein occlusions occur when there is a blockage of veins carrying blood with needed oxygen and nutrients away from the nerve cells in the retina. A blockage in the retina's main vein is referred to as a central retinal vein occlusion (CRVO), while a blockage in a smaller vein is called a branch retinal vein occlusion (BRVO). The narrowed vein experiences turbulent blood flow that promotes clotting, leading to a blockage or occlusion. This obstruction blocks blood drainage and may lead to fluid leakage in the center of vision (macular edema) and ischemia, or poor perfusion (flow) in the blood vessels supplying the macula. Undetected BRVO can lead to permanent blindness.

One embodiment provides a method of treating the symptoms of or inhibiting or reducing the progression of BRVO in a subject in need thereof by topically or intravitreally administering to the subject a composition including PEG-A1 in an amount effective to decrease neovascularization and promote angiogenic repair in the retina. In such an embodiment, PEG-A1 reduces further disease related damage to the retina. In one embodiment, PEG-A1 decreases the expression of inflammatory molecules in the eye. In another embodiment, PEG-A1 increases the production and expression of growth factors in the retina.

4. Age-Related Macular Degeneration

In one embodiment, the disclosed methods and compositions can treat the symptoms of or prevent the progression of age-related macular degeneration (AMD). AMD is the deterioration of the macula, which is the small central area of the retina of the eye that controls visual acuity. It is the leading cause of vision loss among older Americans.

AMD is diagnosed as either dry (non-neovascular) or wet (neovascular). Dry AMD is an early stage of the disease and may result from the aging and thinning of macular tissues, depositing of pigment in the macula, or a combination of the two processes. Dry macular degeneration is diagnosed when yellowish spots known as drusen begin to accumulate in and around the macula. It is believed these spots are deposits or debris from deteriorating tissue. Gradual central vision loss may occur with dry macular degeneration but usually is not nearly as severe as wet AMD symptoms. However, dry AMD through a period of years slowly can progress to late-stage geographic atrophy (GA)—gradual degradation of retinal cells that also can cause severe vision loss.

In about 10 percent of cases, dry AMD progresses to the more advanced and damaging form of the eye disease. With wet AMD, new blood vessels grow beneath the retina and leak blood and fluid. This leakage causes permanent damage to light-sensitive retinal cells, which die off and create blind spots in central vision. Choroidal neovascularization (CNV), the underlying process causing wet AMD and abnormal blood vessel growth, is the body's misguided way of attempting to create a new network of blood vessels to supply more nutrients and oxygen to the eye's retina. Instead, the process creates scarring, leading to sometimes severe central vision loss. Wet macular degeneration falls into two categories occult and classic. In occult AMD, new blood vessel growth beneath the retina is not as pronounced, and leakage is less evident, which typically produces less severe vision loss. In classic AMD, blood vessel growth and scarring have very clear, delineated outlines observed beneath the retina with more severe vision loss.

One embodiment provides a method of treating the symptoms of or reducing or inhibiting the progression of AMD in a subject in need thereof by administering to the subject a composition including PEG-A1 in an amount effective to decrease neovascularization and promote angiogenic repair in the retina. In such an embodiment, PEG-A1 reduces further disease related damage to the macula. In one embodiment, PEG-A1 decreases the expression of inflammatory molecules in the eye. In another embodiment, PEG-A1 increases the production and expression of growth factors in the eye.

In one embodiment, PEG-A1 is prophylactically administered to the eye of a subject having dry-AMD to prevent, reduce, or inhibit the disease from progressing to wet-AMD.

5. Glaucoma

In one embodiment, the disclosed methods and compositions can treat the symptoms of or reduce or inhibit the progression of glaucoma. Glaucoma is a group of eye conditions that damage the optic nerve, and can lead to permanent blindness. The damage to the optic nerve is often caused by abnormally high pressure in the eye. Elevated eye pressure is due to a buildup of aqueous humor that flows throughout the inside of the eye. Aqueous humor normally drains out through the trabecular meshwork at the angle where the iris and cornea meet. However, when fluid is overproduced such as in the case of leaky vessels, the fluid cannot flow out at its normal rate and eye pressure increases. Damage to the optic nerve leads to vision loss. Vision loss due to glaucoma is irreversible. If glaucoma is recognized early, however, vision loss can be slowed or prevented.

One embodiment provides a method of treating the symptoms of or reducing or inhibiting the progression of glaucoma in a subject in need thereof by administering to the eye of the subject a composition including PEG-A1 in an amount effective to decrease neovascularization and promote angiogenic repair in the retina. In such an embodiment, PEG-A1 reduces further disease related damage to the optic nerve. In one embodiment, PEG-A1 decreases the expression of inflammatory molecules in the eye. In another embodiment, PEG-A1 increases the production and expression of growth factors in the eye.

Glaucoma has a genetic component and commonly runs in families. In one embodiment, PEG-A1 is administered prophylactically to a subject that is predisposed to glaucoma to prevent the onset of the disease.

6. Ischemic Retinopathies

In one embodiment, the disclosed methods and compositions can be used to treat or prevent the progression of neurodegeneration as a result ischemic retinopathy. Ischemic retinopathy is a major cause of blindness. Retinal ischemia may result from a number of different causes and may be associated with other diseases and conditions, such diabetes, atherosclerosis, etc. For example, retinal ischemia caused by central retinal vein occlusion (CRVO). CRVO may result from a number of different underlying conditions.

Ischemic retinopathies include a diverse group of retinal diseases in which immature retinal vasculature (e.g., retinopathy of prematurity, incontinentia pigmenti) or damage to mature retinal vessels (e.g., diabetic retinopathy, retinal vein occlusion, sickle cell retinopathy) leads to retinal ischemia. While diverse (and poorly understood) etiologies may lead to insufficient perfusion of the retina, all lead to a common sequela: the formation of abnormal, leaky blood vessels. This can manifest clinically with the accumulation of fluid in the inner retina (i.e., macular edema) and often a profound loss of vision. Indeed, macular edema in patients with ischemia-induced retinopathies remains the leading cause of vision loss in the working-age population in the developed world.

A number of treatments have been suggested for retinal ischemia. For example, to manage outflow obstruction, it has been suggested to administer fibrinolytic agents and anticoagulants, to conduct hemodilution and plasma exchange, to administer steroids, or to conduct photocoagulation (Kohner et al, Ophthalmology, 90(5):484 (1983)). Unfortunately, the aforementioned treatments have not been found very effective (Hansen et al, British Journal of Ophthalmology, 69:108 (1985)).

One embodiment provides a method of treating, reducing, or inhibiting the symptoms of neurodegeneration or reducing or inhibiting the ischemic retonopathy in a subject in need thereof by administering to the subject a composition including PEG-A1 in an amount effective to decrease neurodegeneration and promote neurogenesis in the retina. In one embodiment, PEG-A1 decreases the expression of inflammatory molecules in the eye. In another embodiment, PEG-A1 increases the production and expression of growth factors in the retina.

In one embodiment, a pharmaceutical composition including PEG-A1 is administered to subjects who have been diagnosed with ischemic retinophathy. In such embodiments, PEG-A1 is intravitreally injected into the affected eye or eyes in an amount effective to decrease neurodegeneration and promote neurogenesis in the retina. Administration of PEG-A1 promotes neuroprotection or neurogeneration in the retina by penetrating the blood-retina barrier of a subject diagnosed with ischemic neuropathy.

7. Ischemic Stroke

In one embodiment, the disclosed methods and compositions can be used to treat or prevent the progression of neurodegeneration as a result of an ischemic stroke. Approximately 15 million people worldwide suffer a stroke each year, resulting in death or sensorimotor and other defects. Stroke remains the third most common cause of death in the industrialized world behind heart disease and cancer. There are two forms of stroke: ischemic stroke, caused by a blood clot that blocks or prevents the flow of blood, and hemorrhagic stroke, caused by bleeding into or around the brain. Ischemic stroke accounts for approximately 80-86% of all stroke cases. Current pharmacotherapy for ischemic stroke is limited.

Administration of thrombolytic agents, such as tissue plasminogen activator (tPA), which dissolve blood clots and thus restore blood flow to affected regions, has limited applicability. In particular, administration of tPA is only effective if given within three hours from the time of stroke onset. The use of tPA for treatment of ischemic stroke has other limitations, including that not all clinicians are adequately trained to deliver tPA, and that tPA has also been associated with extravascular deleterious effects, including hemmorhagic transformation, microvascular dysfunction, and excitotoxic neuronal damage.

Moreover, use of thrombolytic agents, such as tPA, as well as other existing stroke therapies, target only a specific subset of deleterious symptoms associated with or resulting from stroke, and therefore fail to provide a complete therapeutic approach for addressing both the immediate and long-term consequences following a stroke.

In another embodiment, the ischemic disorder is cerebrovascular ischemia (e.g., ischemic stroke and transient ischemic attacks), cardiovascular ischemia (e.g., myocardial infarction), peripheral vascular ischemia, renal ischemia and ischemic retinopathy.

Other embodiments provide methods for minimizing functional damage (including stroke-related disability, including speech, movement, memory; improving/treating paralysis; improving cognitive function/reducing cognitive impairment, etc.) in a subject, or repairing such damage, including restoring the subject to normal state. The extent of stroke-related disability or functional damage and any improvement or restoration of function can be assessed using any functional assessment known to one of skill in the art. Examples of functional assessment methods include Berg Balance Scale, Modified Rankin Scale, Stroke Impact Scale, and Stroke Specific Quality of Life Measures.

One embodiment provides a method of treating the symptoms of or reducing or inhibiting neurodegeneration as a result of an ischemic stroke in a subject in need thereof by systemic administration of a composition including PEG-A1 in an amount effective to decrease neurodegeneration and promote neurogenesis to repair in the brain. In such an embodiment, PEG-A1 reduces further disease related ischemic damage to the brain. Administration of PEG-A1 promotes neuroprotection or neurogeneration in the brain by penetrating the blood-brain barrier of a subject diagnosed with ischemic stroke.

In some embodiments, the subject will be one that has had a stroke before the stroke with which the present disclosure is concerned. Such subjects will often be on secondary preventative measures to reduce the risk of further strokes or susceptibility to greater damage upon occurrence of an ischemic event. Administration of such agents in simultaneous, separate, or sequential administration with PEG-A1 is specifically contemplated.

8. Traumatic Optic Neuropathy

In one embodiment, the disclosed methods and compositions can treat neurodegeneration in a subject is the result of acute retinal injury, wherein the acute retinal injury results in traumatic optic neuropathy (TON). TON is rare type of optic neuropathy involving loss of vision following damage to the optic nerve secondary to traumatic injury. TON frequently results in profound loss of central vision with the final visual outcome largely dictated by the patient's baseline visual acuities. TON is classified by either the site or mode of traumatic injury. Exemplary sites of injury that lead to TON include trauma to the optic nerve, head trauma, intraorbital injury, intracanalicular injury, and/or intracranial injury.

One embodiment provides a method of treating the symptoms of or reducing or inhibiting the TON in a subject in need thereof by administering to the eye of the subject a composition including PEG-A1 in an amount effective to decrease neurodegeneration and promote neurogenesis to repair in the retina. In such an embodiment, PEG-A1 reduces further disease related damage to the retina.

### EXAMPLES

### Example 1. A1 Deletion Inhibits Vascular Repair and Increases Pathological Angiogenesis During the Hypoxia Phase of OIR

Materials and Methods:

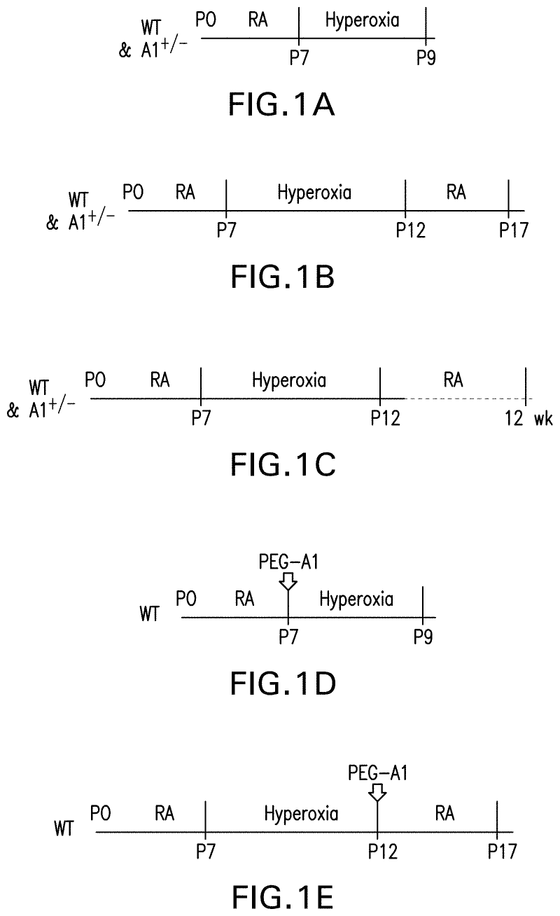

OIR mouse model: Wild type (WT) or heterozygous A1 knock out (A1^{+/-}) mice and their wild type (WT) littermates were subjected to oxygen induced retinopathy (OIR). Because deletion of both copies of A1 is lethal due to hyperammonemia, mice lacking 1 copy of A1 were used which is sufficient to dampen its activity (Zhang, et al., Am J Pathol, 175:891-902 (2009); Elms, et al., Diabetologia, 56:654-662 (2013); Patel, et al., Front Immunol, 4:173 (2014)). OIR was induced in newborn mice according to the protocol of Smith, et al. with some adjustments (Connor, et al., Nature Protocols, 4:1565-1573 (2009)). On P7 (postnatal day 7), mice were placed along with their dams in a hyperoxia (70% oxygen) chamber for up to 5 days, after which they were transferred back to room air on P12. The 70% oxygen concentration was used for experiments involving A1^{+/-} mice based on preliminary experiments showing intolerance of the A1^{+/-} mice to 75% oxygen treatment. Mice were sacrificed at various times and retinas were collected for analyses (FIG. 1A-1C). In all experiments, mice were compared to their littermate controls. Then data were pooled together to minimize variability between litters due to differences in litter size and weight gain (Kim, et al., Eye and Brain, 8:67-79 (2016)). These experiments were performed in accordance with the ARVO Statement for the Use of Animals in Ophthalmic and Vision Research, and were approved by the institutional animal care and use committee.

Western blotting analysis: Retina lysates were homogenized in RIPA buffer supplemented with protease and phosphatase inhibitors. Samples (20 μg protein) were run on 10% sodium dodecyl sulfate-polyacrylamide gel electrophoresis, transferred to nitrocellulose membrane and reacted with primary antibody followed by horseradish peroxidase-linked secondary antibody and enhanced chemiluminescence (Amersham Pharmacia). β-actin (mouse monoclonal antibody, 1:5000, Sigma-Aldrich) or GAPDH served as a loading control. The following primary antibodies were used: GFAP, PARP, A1.

Analysis of vaso-obliteration and neovascularization: Eyeballs were fixed in 4% paraformaldehyde (PFA) overnight. Retina flat-mounts were dissected and then blocked and permeabilized in phosphate-buffered saline (PBS) containing 10% goat serum and 1% Triton X-100 (Sigma-Aldrich) for 30 minutes. Retinas were then immunolabeled with Alex594-labeled Griffonia simplicifolia isolectin B4 (1:200; Invitrogen, Carlsbad, Calif.) overnight at 4° C. Retinas were flat-mounted in mounting medium (Vectashield; Vector Laboratories, Burlingame, Calif.) and images were captured using fluorescence microscopy (Axioplan 2, Carl Zeiss Vision Inc.). Areas of central capillary dropout (vaso-obliteration, VO) and vitreoretinal neovascular tufts (neovascularization, NV) were quantified in a masked fashion using ImageJ software (NIH, Bethesda, Md.) (Connor, et al., Nature Protocols, 4:1565-1573 (2009); Patel, et al., Am J Pathol, 184:3040-3051 (2014)). High-magnification images were acquired to perform vessel sprouting analysis. Vessel sprouts were counted as previously described Patel, et al., Am J Pathol, 184:3040-3051 (2014)).

Statistical analysis: Statistical analysis was conducted using GraphPad Prism 7 software. Differences between 2 groups were determined by student's t-test. Comparisons between multiple groups were analyzed by ANOVA with Tukey's post-hoc test. P values <0.05 were considered as statistically significant. Graphs were prepared using GraphPad Prism 7 software and data were presented as mean±standard deviation (SD) or as mean±standard error (SE).

Results:

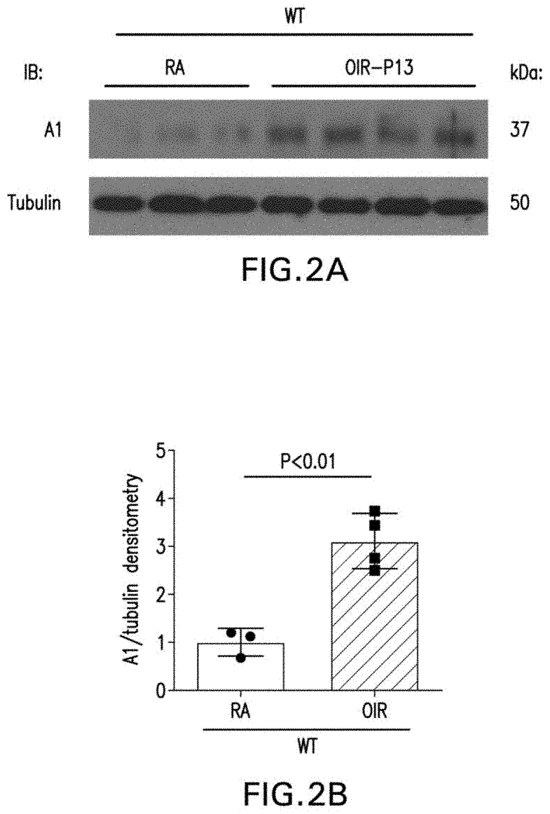

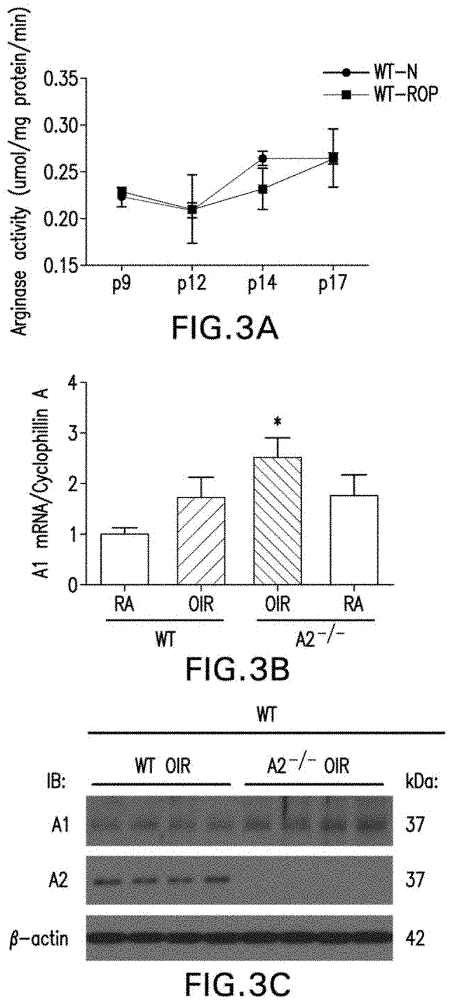

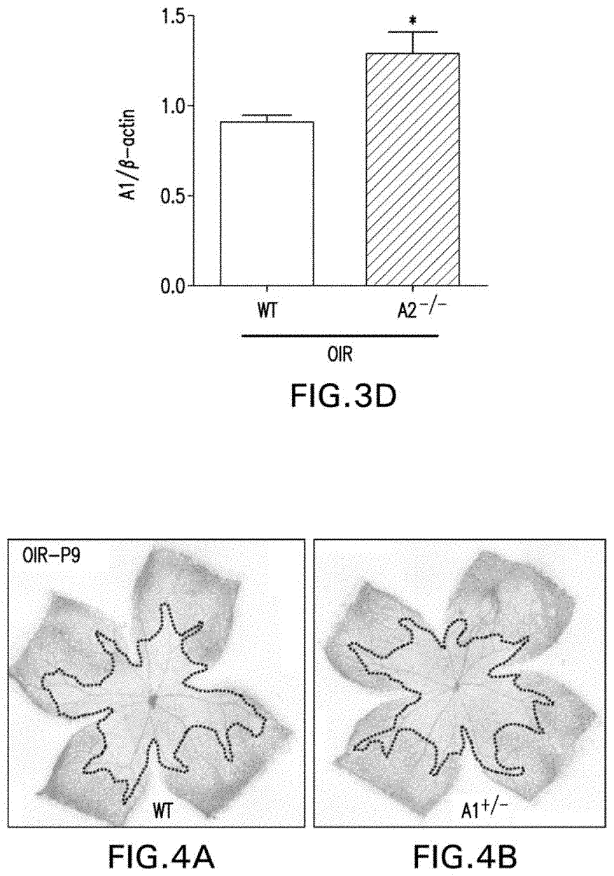

Previous studies in knockout mice lacking the A2 isoform of arginase have shown that vaso-obliteration is reduced during the hyperoxia phase of OIR, whereas vascular repair is enhanced and pathological angiogenesis is reduced during the hypoxia phase (Suwanpradid, et al., PLOS One, 9:e110604 (2014)). These protective effects of the A2 deletion were abrogated in double knockout (KO) mice that lacked one copy of the A1 isoform as well as both copies of A2 (A1^{+/-}A2^{-/-}). Western blot analysis of A2^{-/-} retinas following hyperoxia treatment showed that the protective effects of the A2 deletion are associated with an increase in A1 expression (FIG. 3B-3D) suggesting that A1 has a protective role in the vascular pathology. A1^{+/-} mice and their littermate controls were subjected to OIR and sacrificed at various time points to examine the effect of A1 deletion in both the vaso-obliteration and neovascularization phases of the model. A1 is increased in the WT retina after OIR as evidenced by Western blot (FIGS. 2A-2B).

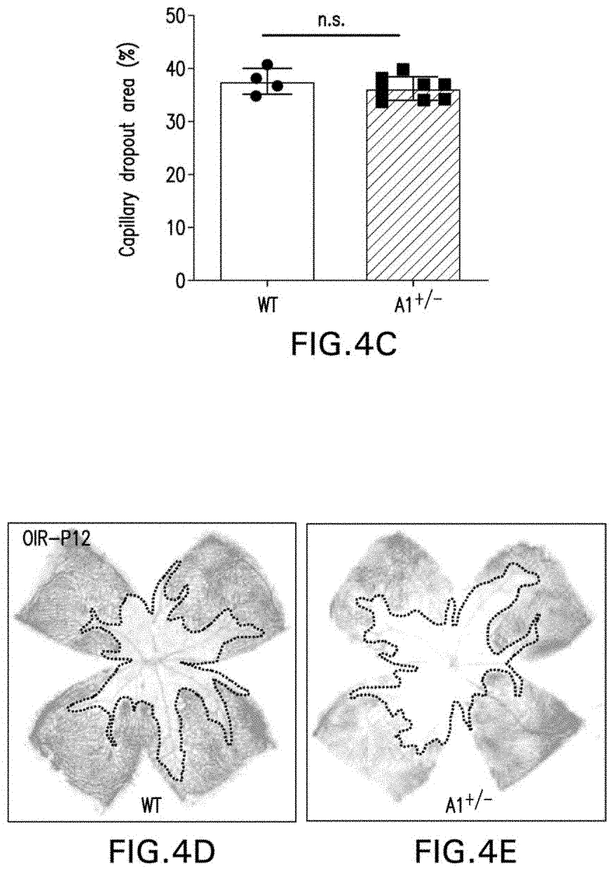

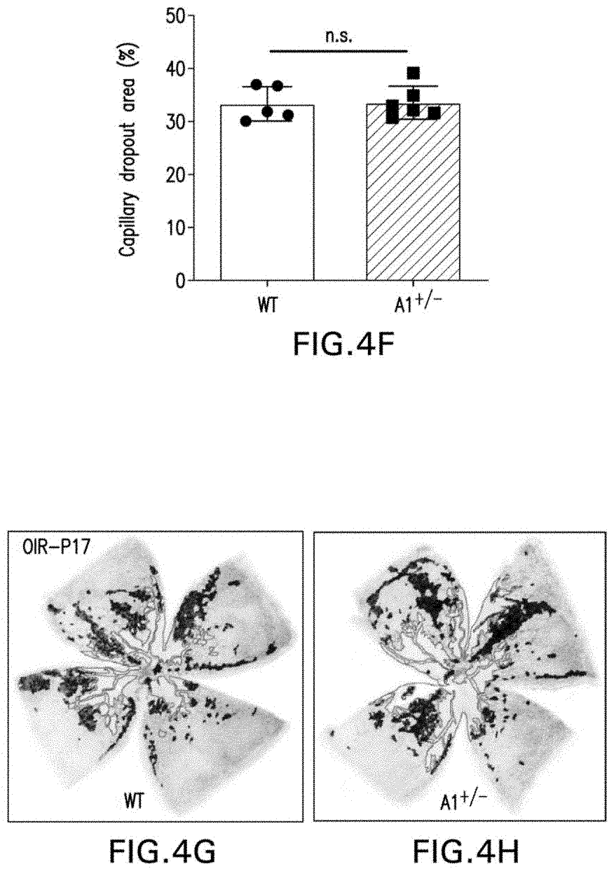

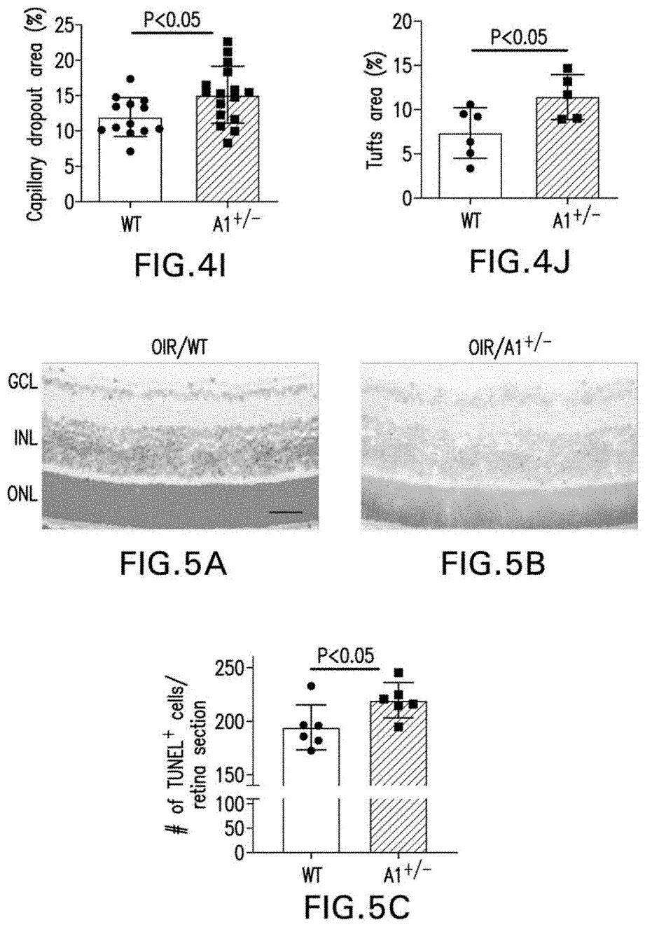

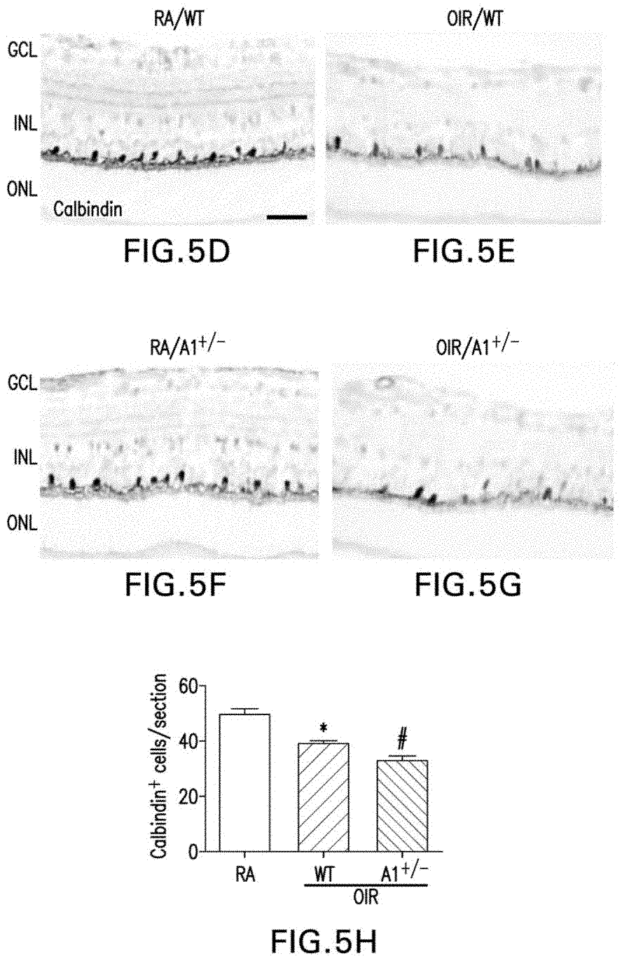

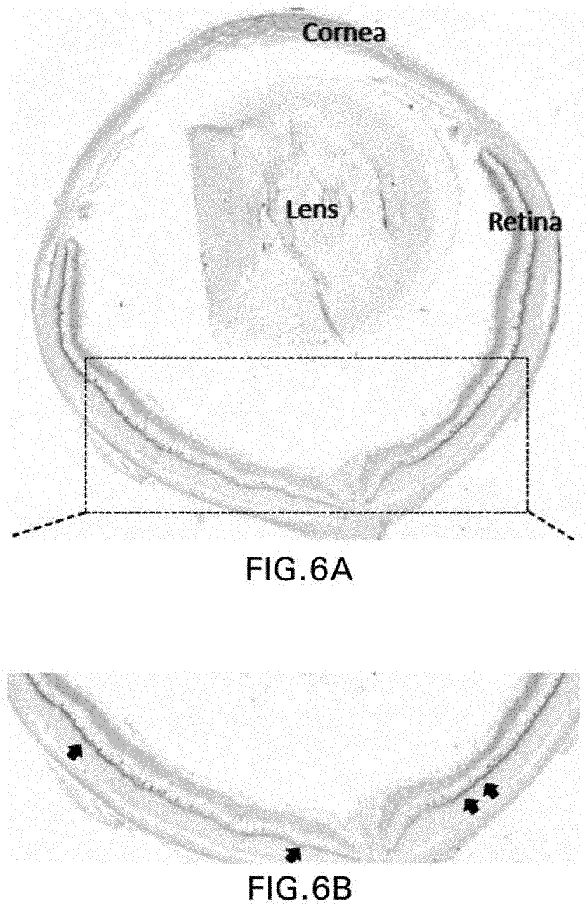

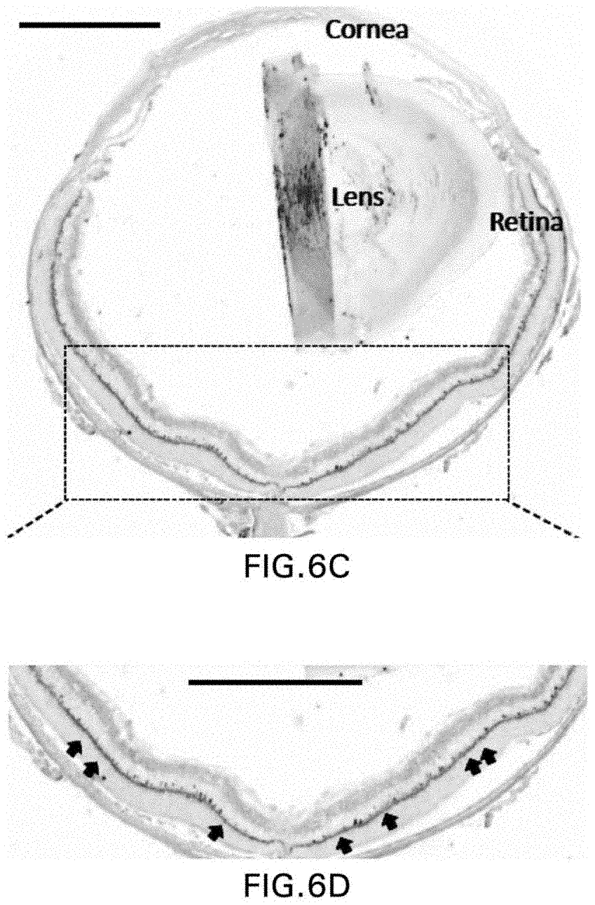

Morphometric analyses of isolectin B4-labeled retinal flatmounts prepared after either 2 days (FIGS. 4A-4C) or 5 days (FIGS. 4D-4F) of hyperoxia treatment showed no effect of the A1 deletion on vaso-obliteration. In order to determine the effects of A1 deletion on pathological neovascularization and physiological vascular repair, A1^{+/-} KO mice and their WT littermate controls were maintained in hyperoxia from P7 to P12 and returned to normoxia for 5 days until P17. Morphometric analysis of lectin-labeled retinal flat mounts showed significant increases in the areas of both capillary drop-out and preretinal neovascular tuft formation in the A1^{+/-} mice as compared to the WT littermate controls (FIG. 4G-4I), indicating a role for A1 in limiting the vascular pathology. A1 deletion increases TUNEL positive cells after OIR (FIGS. 5A-5C). In addition, A1 deletion increases horizontal cell loss after OIR (FIGS. 5D-5H, 6A-6D). Taken together, these results suggest that A1 plays no role in OIR-induced vaso-obliteration but that A1 expression is involved in promoting reparative angiogenesis and limiting pathological neovascularization during the second phase of OIR (FIGS. 4G-4H, 4J).

Unlike A2, A1 deletion had no effect on the avascular area in the vaso-obliteration phase of OIR as measured at P9 (maximum avascular area) and P12 (end of the vaso-obliteration phase). In the neovascularization phase of OIR, A1 is upregulated 1 day after switching the pups to room air. This endogenous upregulation is protective since A1 deletion increased pathological angiogenesis and vitreoretinal neovascular tufts formation while A1 treatment enhanced physiological angiogenesis.

### Example 2. A1 Deletion Worsens OIR-Induced Glial Activation and Retinal Thinning

Materials and Methods:

Immunofluorescence labelling: PFA fixed eyeballs were washed in PBS and cryoprotected. Cryostat sections (15 μm) were permeabilized in 1% Triton (20 min) and blocked in 10% normal goat serum containing 1% BSA (one hour). Sections were then incubated overnight in primary antibodies at 4° C. On day two, the sections were incubated at room temperature for 1 hour in fluorescent conjugated secondary antibodies (Life Technologies), washed in PBS and mounted with Vectashield (Vector Laboratories)

Results:

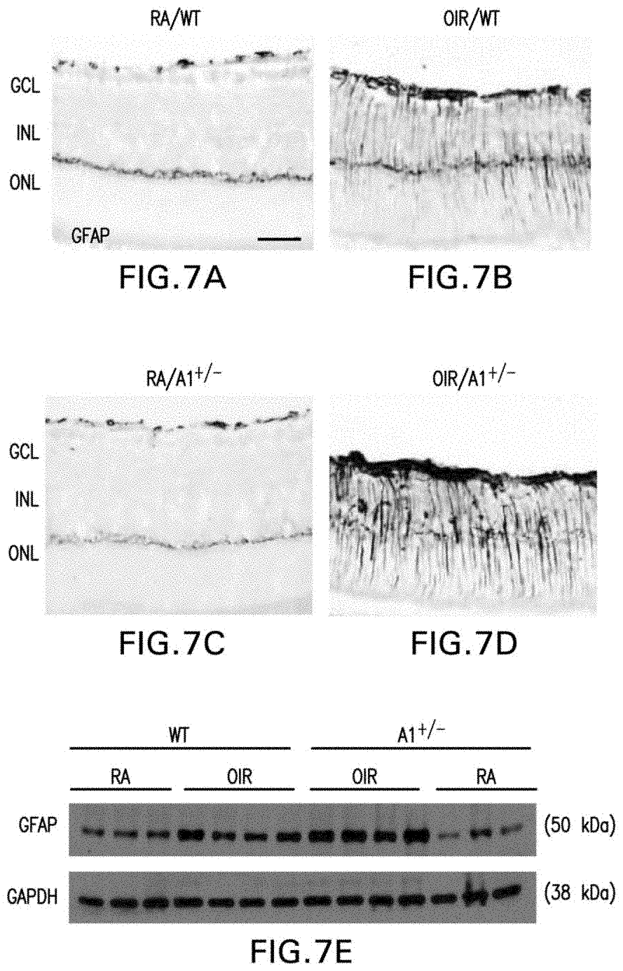

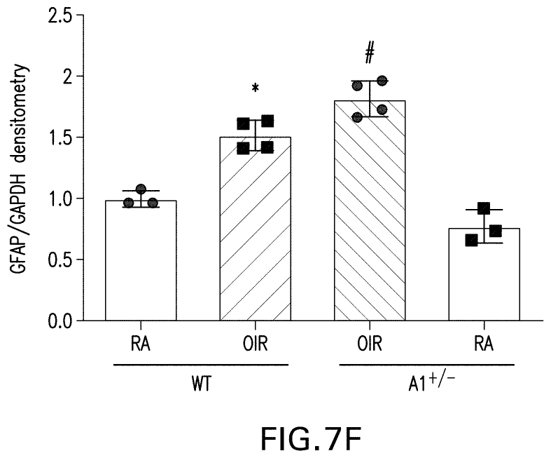

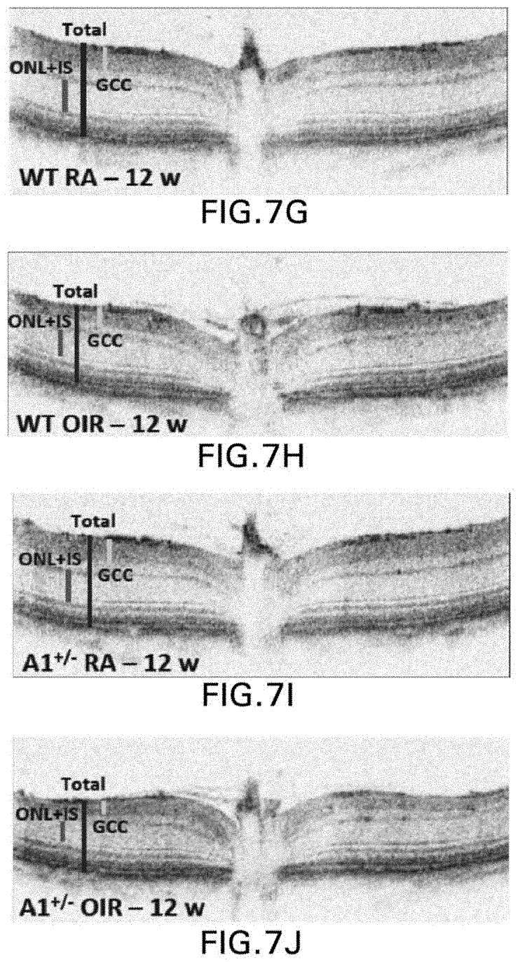

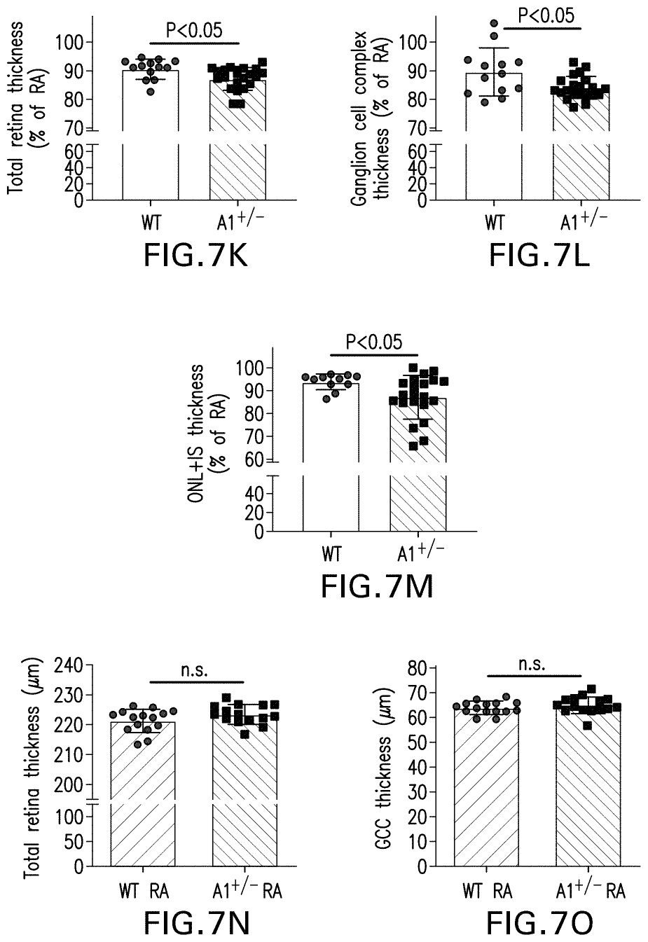

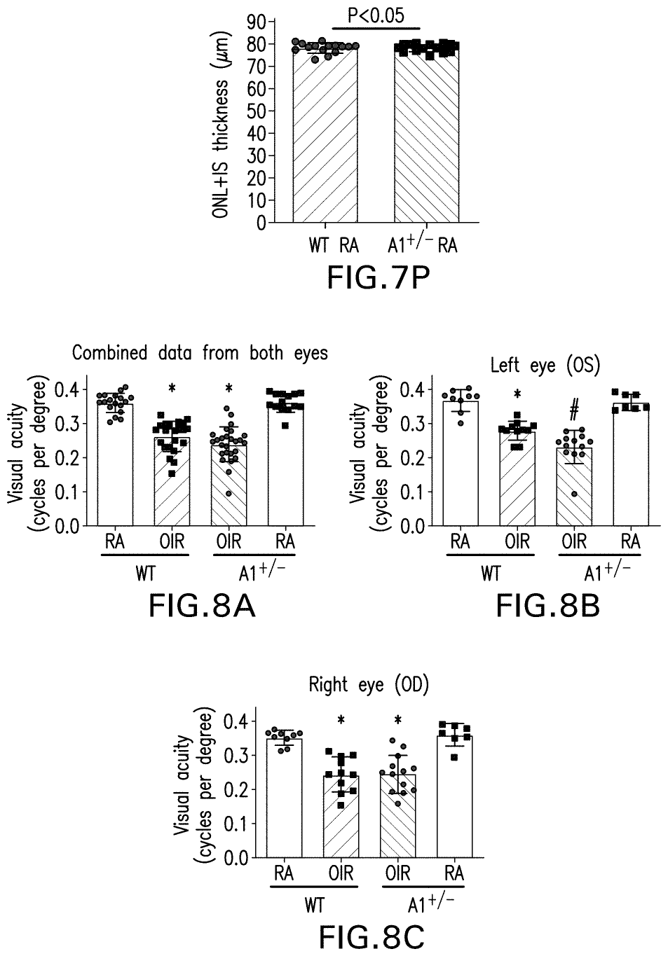

To examine Müller cell activation which is a prominent feature of OIR-induced retinal injury (Narayanan, et al., PLOS One, 6:e110604 (2014)), immunolabelling was performed on retina cross-sections as well as western blotting on retina tissue lysates using anti-glial fibrillary acidic protein (GFAP) antibody. Compared to WT retinas, A1^{+/-} retinas showed increased Müller cell activation as evidenced by GFAP immunolabelling (FIGS. 7A-7D) as well as western blotting at P17 (FIGS. 7E-7F).

Chronic morphological changes and retinal thinning have been previously reported in mice subjected to OIR (Nakamura, et al., PLOS One, 7:e32167 (2012)). Retinal thickness was determined at 12 weeks in WT and A1^{+/-} mice subjected to OIR by using spectral domain optical coherence tomography (SD-OCT). This analysis showed a significant worsening of retinal thinning in A1^{+/-} mice as compared to the WT OIR group. This was evident in the total retina thickness, ganglion cell complex (GCC) thickness and outer nuclear layer plus inner segments (ONL+IS) thickness (FIGS. 7G-7M). Thickness of retinal layers was not different among the room air groups (FIGS. 7N-7P).

Taken together, the results in A1^{+/-} retinas show amplification of the pathological vascular response to OIR. Moreover, the vascular alterations are accompanied by glial activation together with retinal cell loss and thinning.

### Example 3. A1 Treatment Promotes Reparative Angiogenesis in OIR and Decreases Pathological Neovascularization

Materials and Methods:

PEG-A1 treatment in OIR: PEGylated-A1 (PEG-A1) was prepared from a 3.4 mg/mL stock by dilution in PBS (1:250 ratio) to achieve final concentration of 13.6 ng/μL. PBS was used as vehicle control. Pups were anesthetized before treatment by intraperitoneal (i.p.) injection of ketamine/xylazine mixture. Intravitreal injections were performed using a 36-gauge NanoFil needle mounted to a 10-μL Hamilton syringe (World Precision Instruments). Two treatment strategies were employed. To examine vaso-obliteration, wild-type (WT) pups received intravitreal injection of PEG-A1 (6.8 ng in 0.5 μL—based on a preliminary dose/response study) at P7 then subjected to hyperoxia (75% oxygen) for 2 days and sacrificed at P9. The P9 time point was selected based on the fact that vaso-obliteration occurs within the first 48 hours of hyperoxia treatment (Connor, et al., Nature Protocols, 4:1565-1573 (2009); Lange, et al., Graefe's archive for clinical and experimental ophthalmology, 247:1205-1211 (2009); Gu, et al., Investigative ophthalmology & visual science, 43:496-502 (2002)). Another cohort of WT pups was placed in hyperoxia (75% oxygen) on P7, switched to room air on P12, immediately given intravitreal PEG-A1 injections (6.8 ng in 0.5 μL), and sacrificed on P17 (FIG. 1D-1E).

Results:

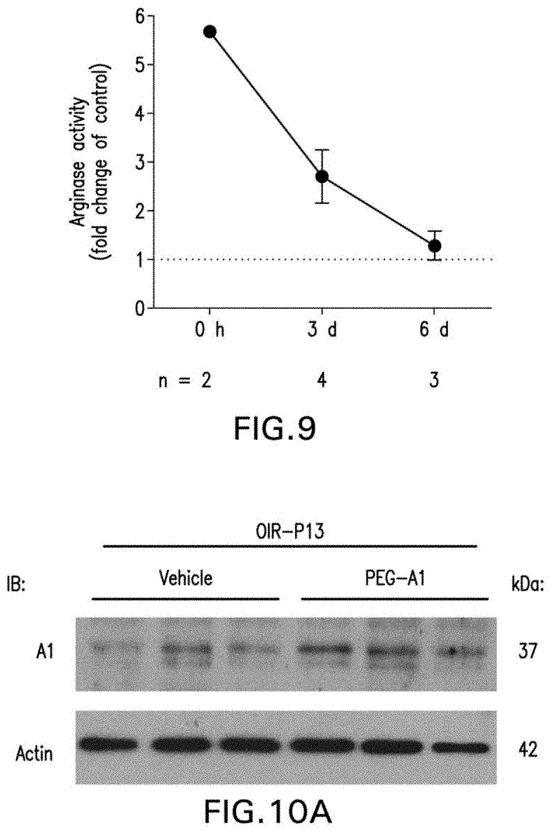

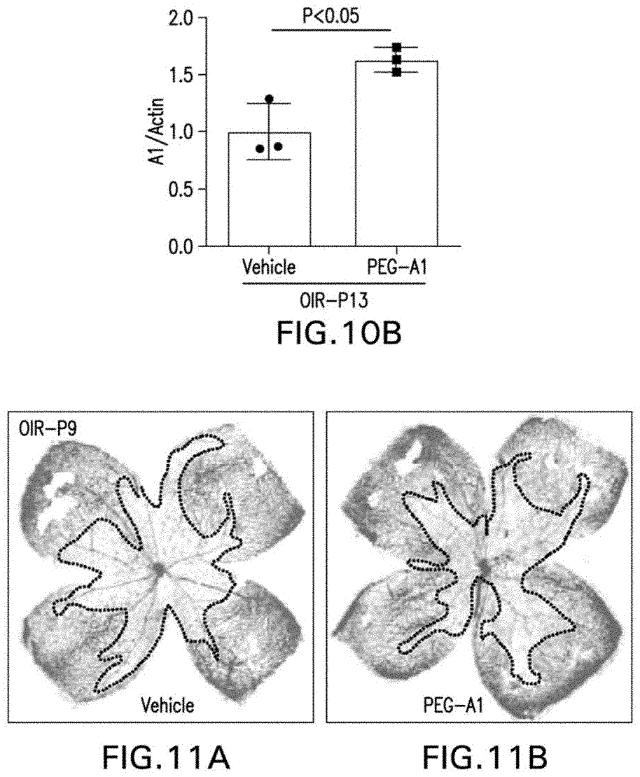

Based on the results of the above studies showing detrimental effects of A1 hemizygous deletion on vascular repair in A1^{+/-} mice and the findings that increases in A1 expression accompany the beneficial effects of A2 deletion on the vascular injury, it was hypothesized that treatment with A1 could be useful in limiting OIR-induced retinal injury. To test this, a PEG-A1 was used. First, the pharmacokinetic profile of PEG-A1 in the mouse vitreous was established by measuring the arginase activity after PEG-A1 intravitreal administration (6.8 ng in 1 μL). Adult mice were used in this experiment to get higher vitreous yield for ease of processing. Immediately after intravitreal administration, vitreous arginase activity reached about six-fold increase as compared to vehicle treatment. Arginase activity stayed high (three-fold at day 3 post injection) and returned to baseline at day 6 (FIG. 9). A1 protein expression is increased in OIR mice treated with PEG-A1 (FIGS. 10A-10B).

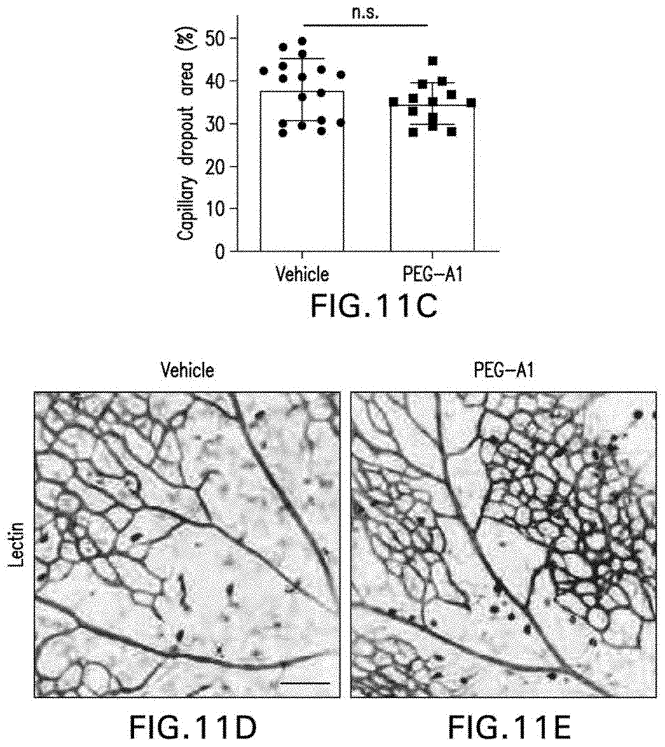

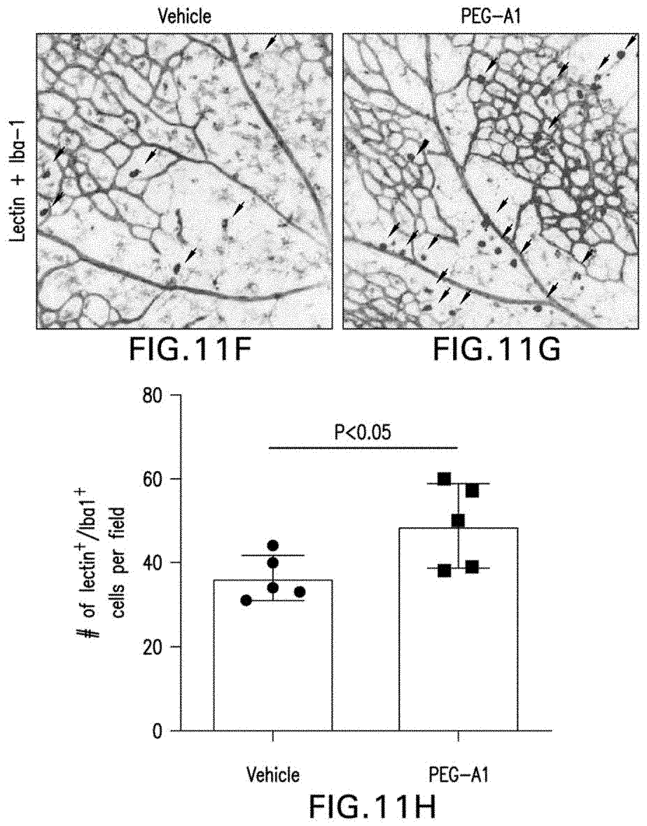

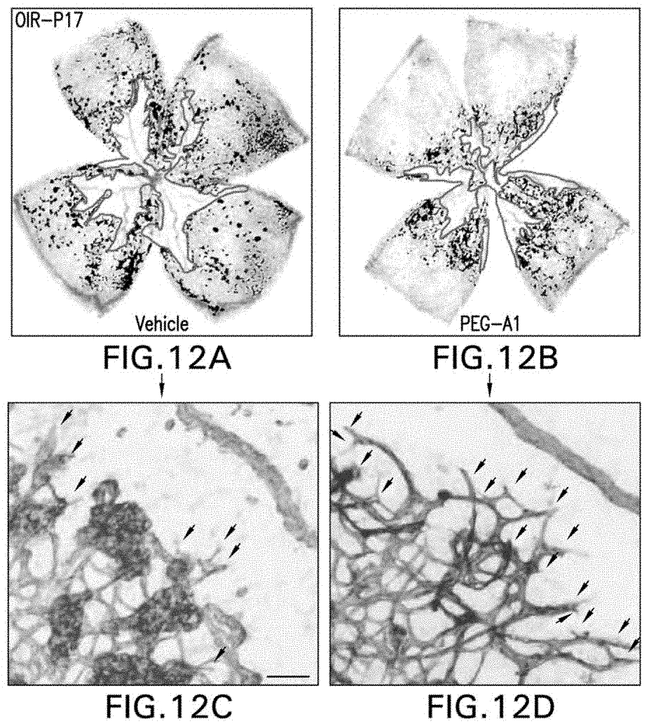

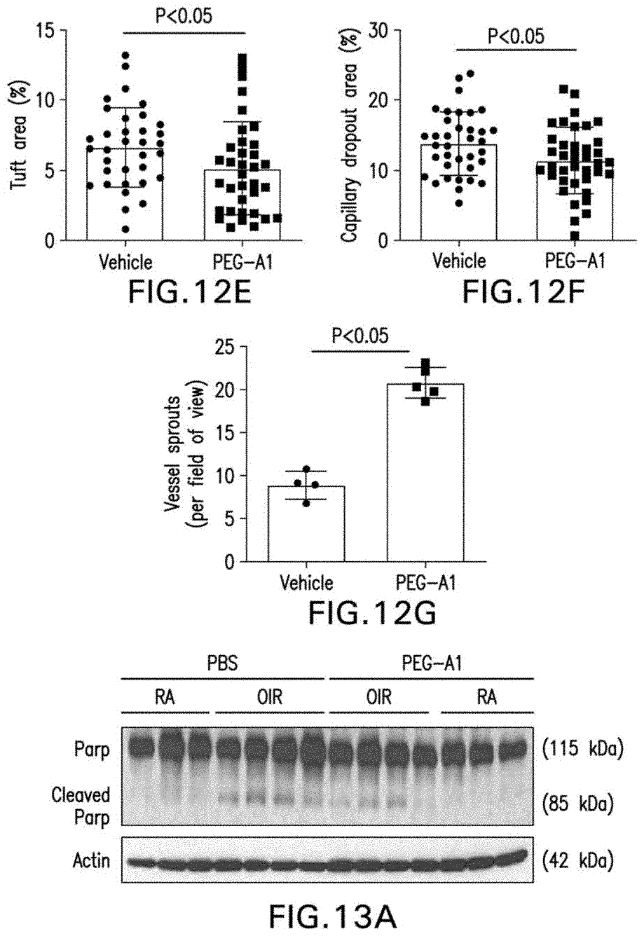

The effects of the PEG-A1 treatment on OIR induced retinal vascular injury were determined. WT pups were subjected to 5 days of hyperoxia starting at P7 then switched to room air at P12 and treated with intravitreal PEG-A1 (6.8 ng in 0.5 μL, this dose was selected based on preliminary studies). PEG-A1 treatment does not affect hyperoxia-induced vaso-obliteration (FIGS. 11A-11C). PEG-A1 treatment increased lectin-positive vitreal macrophages after OIR (FIGS. 11D-11H). Morphometric analysis at P17 showed reduced neovascular tufts in the PEG-A1 treated retinas, thus highlighting a decrease in pathological neovascularization (FIGS. 12A-12G). Moreover, avascular area was reduced with PEG-A1 treatment suggesting an increase in reparative angiogenesis leading to revascularization (FIGS. 12A-12B, 12F). In accordance with this, the PEG-A1 treatment resulted in a two-fold increase in endothelial tip cells (vessel sprouts) per field of view (FIGS. 12C-12D, 12G).

### Example 4. A1 Treatment Protects Against OIR-Induced Apoptosis, Horizontal Cells Loss, and Retinal Thinning

Results:

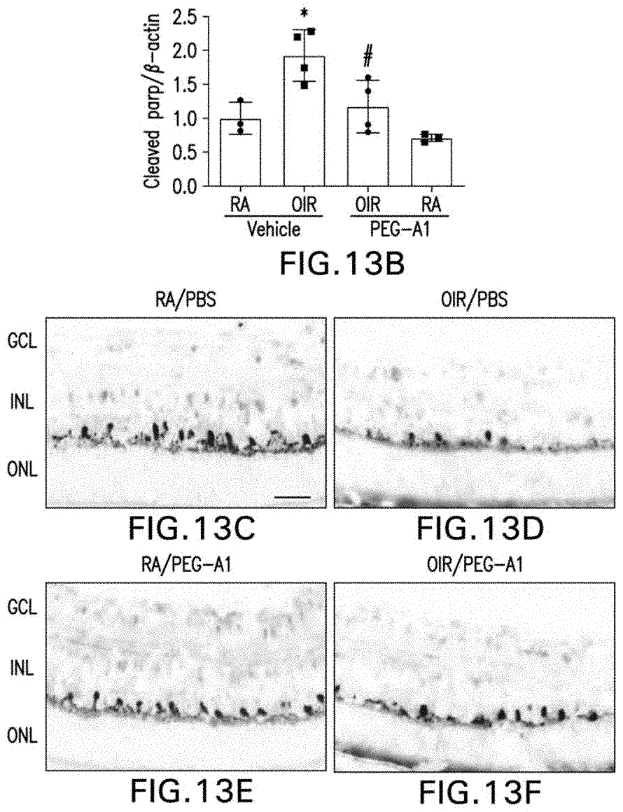

Western blotting and quantification (FIGS. 13A-13B) on P17 OIR retinal lysates showed cleavage of the pro-apoptotic marker, PARP, which was significantly reduced with PEG-A1 treatment. Furthermore, the effect of PEG-A1 treatment on horizontal cell loss was examined using calbindin immunolabelling. PEG-A1 treatment significantly rescued the OIR-induced calbindin-positive horizontal cell loss at P17 as compared to vehicle treatment (FIGS. 13C-13G).

### Example 5. A1 Treatment Increases Retina Growth Factors and Ameliorates OIR-Induced Inflammatory Response

Results:

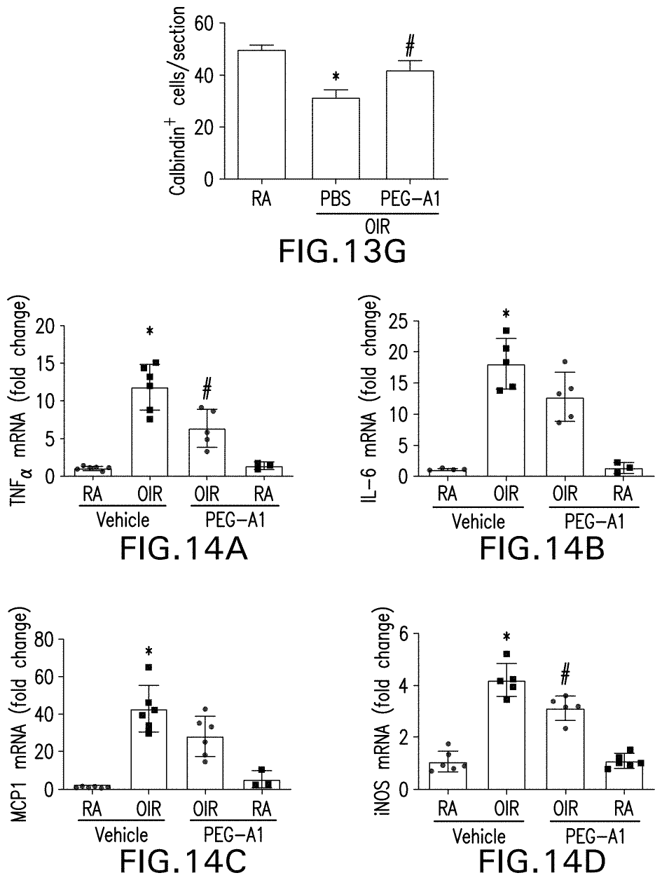

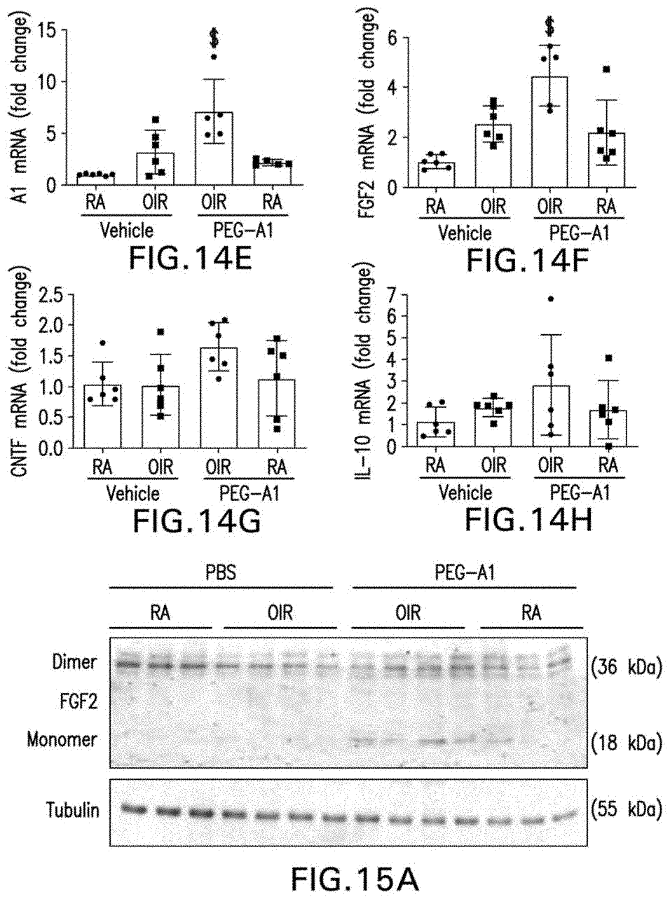

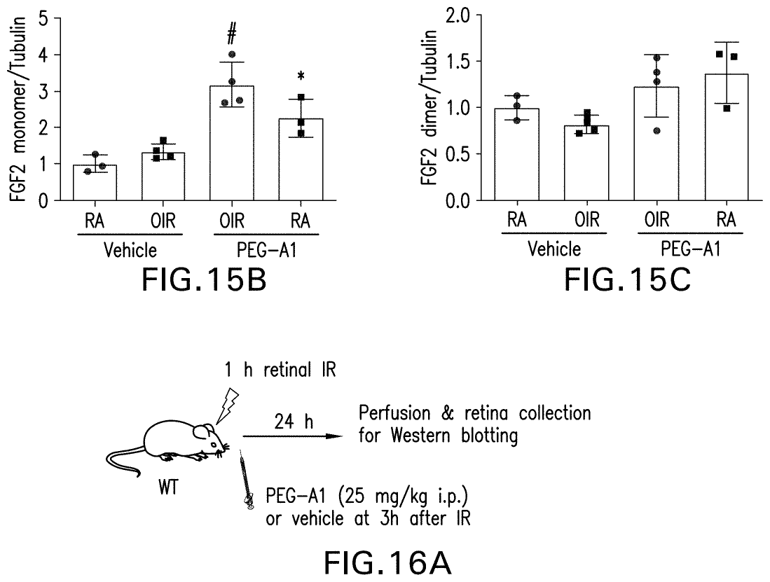

To examine the effect of PEG-A1 on OIR-induced inflammation markers, qPCR was performed on retina samples collected from PEG-A1 or vehicle treated mice and sacrificed at P13 or P17. OIR retinas showed upregulation of the inflammatory markers, tumor necrosis factor (TNF) α, interleukin (IL-) 6, and MCP1 (Monocyte chemoattractant protein 1) (FIGS. 14A-14C). PEG-A1 treatment reduced the expression of these inflammatory markers. However, variability was relatively high in the OIR retinas and this protective effect was statistically significant only for TNFα. OIR also increased mRNA levels of inducible nitric oxide synthase (iNOS) in WT retinas and this was significantly reduced with PEG-A1 treatment (FIG. 14D). Furthermore, PEG-A1 treated OIR retinas showed mRNA upregulation of A1, the growth factors, fibroblast growth factor 2 (FGF2) and ciliary neurotrophic factor (CNTF), and the anti-inflammatory cytokine, IL-10, yet the latter two did not reach statistical significance (FIGS. 14E-14H, 15A-15C).

### Example 6. Systemic PEG-A1 Treatment Crosses the Blood-Retina Barrier and Provides Neuroprotection after IR

Materials and Methods:

Mouse retinal ischemia-reperfusion (IR) injury and traumatic optic neuropathy (TON) models: All experiments were performed in accordance with the ARVO Statement for the Use of Animals in Ophthalmic and Vision Research and were approved by the institutional animal care and use committee (Animal Welfare Assurance no. D16-00197). Wild-type (WT) C57BL/6J mice (10-12 weeks old) were anesthetized using ketamine/xylazine mixture then subjected to retinal ischemia-reperfusion (IR) injury or optic nerve crush (ONC) to model traumatic optic neuropathy (TON) as described in Shosha, et al., Cell death & disease, 7:e2483-e (2016) and Xu, et al., Frontiers in Neuroscience, 12:970 (2018). Mice were sacrificed at 24 hours or 7 days after by deep anesthesia followed by trans-cardial perfusion with PBS or thoracotomy and eyeballs were collected and fixed in 4% PFA for immunolabeling or retinas were extracted without fixation and homogenized in Radioimmunoprecipitation Assay (RIPA) lysis buffer for Western blotting.

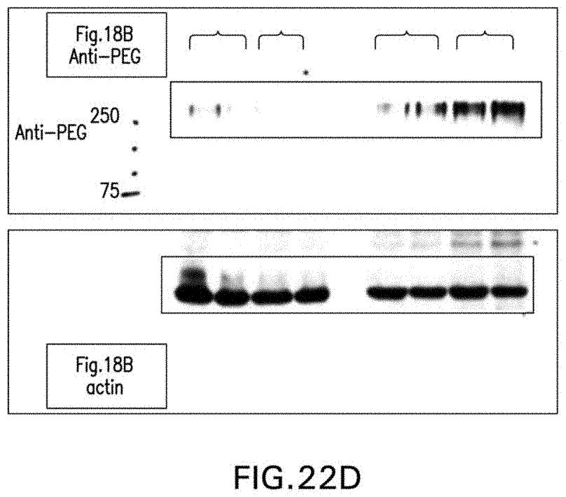

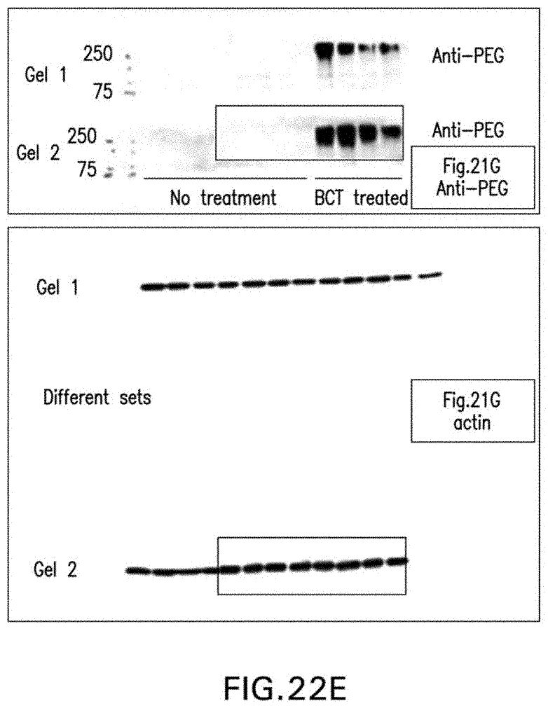

Western blotting: Western blotting on retina or brain was conducted as described in Fouda, A Y, et al., Cell death & Disease, 9:1001 (2018). Membranes were probed with anti-PEG (RevMAb Biosciences, 1:1000) and anti-albumin (Bethyl Laboratories, 1:5000). Uncropped blots are included in FIG. 22.

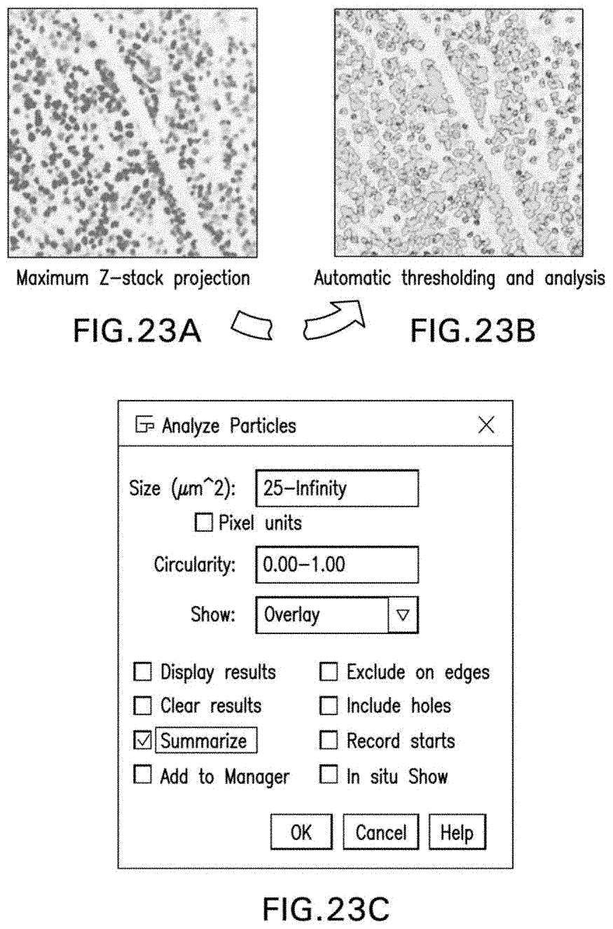

Evaluation of retinal neurodegeneration: Retina flat-mounts and explants were immunolabeled for the neuronal marker, NeuN (Millipore), as previously described in Shosha, et al., Cell death & disease, 7:e2483-e (2016). Images were collected and analyzed in a blinded fashion using a semi-automated method. Four images per retina flat-mount were taken as Z-stacks in the mid-periphery of each retina petal using an inverted confocal microscope (LSM 780; Carl Zeiss). Three images were taken per explant. NeuN-positive cells were quantified using ImageJ software ‘Batch processing’ function. A prerecorded ImageJ macro code (FIG. 23A-23C) was used for batch processing. The macro was validated every time it was used to make sure it is compatible with the set of images being analyzed. Results from three to four images were averaged to represent neuron density for each retina. Results are presented as % of sham retina or control explant under normoxic conditions.

Fluorescent immunolabeling: Retinal flat-mounts were permeabilized and immunolabeled with anti-PEG antibody (RevMAb Biosciences, 1:400) and co-stained with Alex594-labeled Griffonia simplicifolia isolectin B4 (1:200; Invitrogen) as described in Shosha, et al., Cell death & disease, 7:e2483-e (2016).

Results:

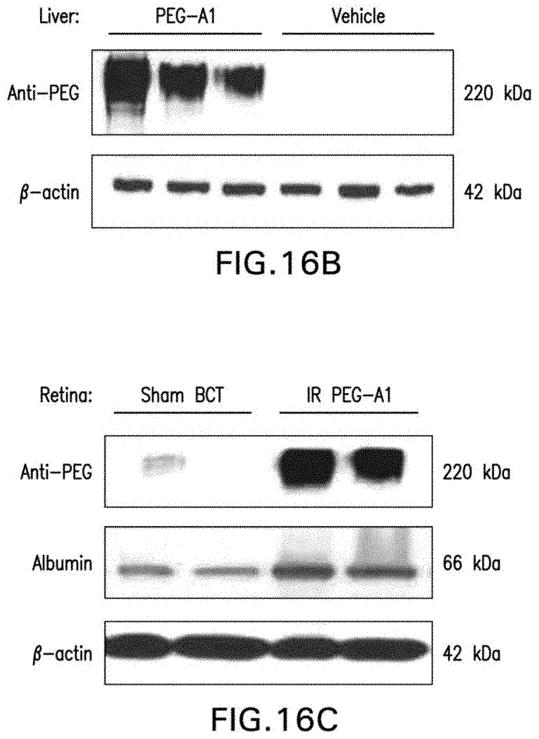

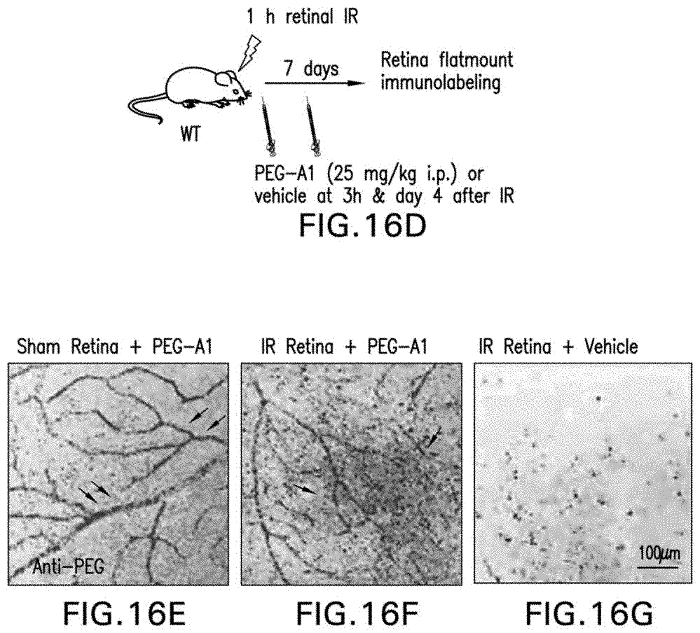

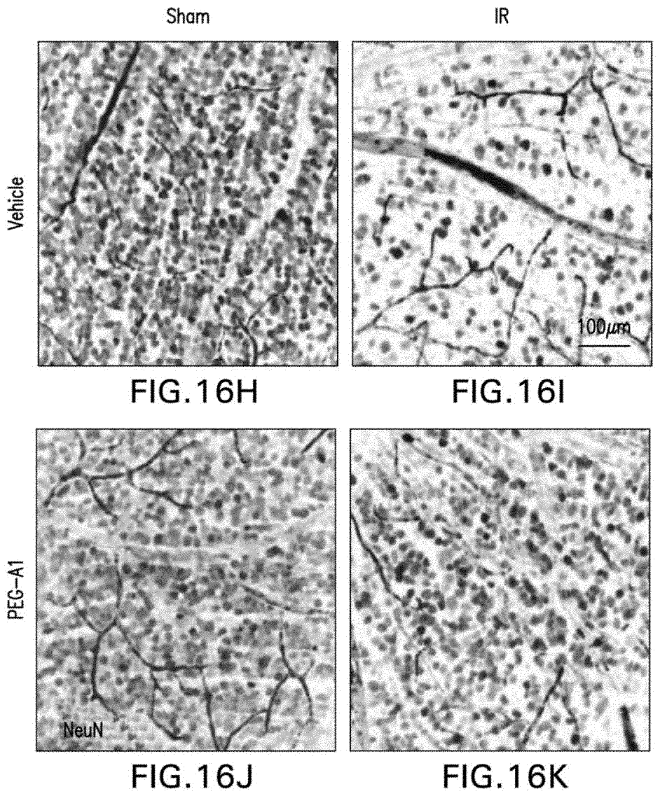

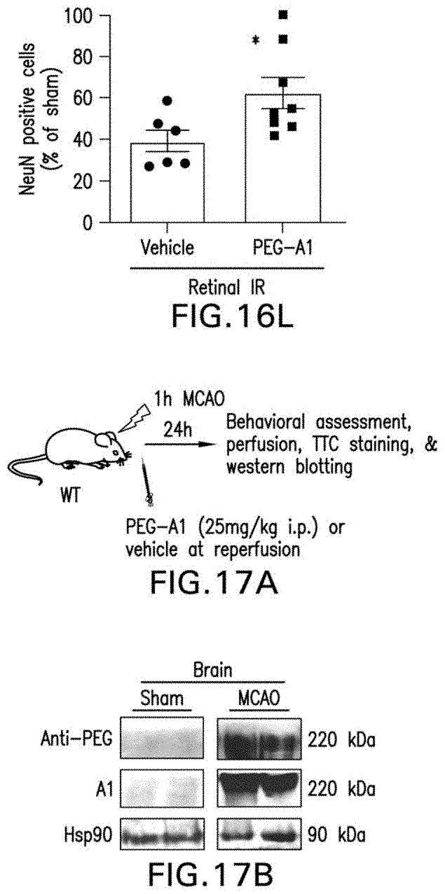

To examine the PEG-A1 penetration of the blood-retina barrier with systemic treatment, acute retinal ischemic injury model mice were subjected to 1 hour of unilateral retinal ischemia-reperfusion injury and injected with PEG-A1 (25 mg/kg, i.p.) at 3 hours after reperfusion. Mice were deeply anesthetized and sacrificed 24 hours later by trans-cardial perfusion with PBS (FIG. 16A). Western blotting showed a strong band for anti-PEG immunoreactivity in liver homogenates of PEG-A1 treated mice (FIG. 16B). Retina homogenates from the sham eyes of treated mice showed faint anti-PEG reactive bands while the IR-injured retinas from the same mice showed strong anti-PEG reactivity as well as increased levels of albumin leakage, a measure of the IR-induced increase in permeability of the blood-retinal barrier (FIG. 16C). Another cohort of WT mice were subjected to sham or IR injury and treated with PEG-A1 (25 mg/kg, i.p.) at 3 hours after reperfusion and at day 4. These mice were sacrificed at day 7 without trans-cardial perfusion and retina flat-mounts were immunolabeled to show PEG distribution within the retina (FIG. 16D). In the sham retinas, the PEG signal was restricted to a vessel-like network. The IR-injured retinas showed a more diffuse signal, suggesting extravasation of PEG-A1 had occurred after the injury (FIG. 16E-16G). Taken together, these results show an enhanced penetration of PEG-A1 after blood-retina barrier breakdown. Furthermore, mice treated with systemic PEG-A1 showed increased neuronal survival (62% vs 40% in the vehicle group) after retinal IR compared to the vehicle treated mice as measured by NeuN labeling of retinal flat-mounts at 7 days post-injury (FIG. 16H-16K, 16L).

### Example 7. Systemic PEG-A1 Treatment Crosses the Blood-Brain Barrier and Provides Neuroprotection after MCAO

Materials and Methods:

MCAO model: C57BL/6J mice (12-16 week-old male, 25-30 g) were subjected to middle cerebral artery occlusion (MCAO) using the intraluminal filament technique (Docol 602145) for 60-minutes followed by reperfusion as described in Eldahshan, et al., American Journal of Physiology Heart and Circulatory Physiology, 316:H1192-h201 (2019). Animals that did not show a motor deficit after stroke were excluded from the study. A heating pad was used to maintain body temperature at 37° C. Animals showed 12 to 14% weight loss at 24 hours which was not different between the groups. Sham surgery involved all the regular surgery steps except the filament insertion. Mice were deeply anesthetized at 24 hours and sacrificed by trans-cardial perfusion with PBS then brains were collected and sectioned for triphenyl tetrazolium chloride (TTC) staining or homogenized in RIPA lysis buffer for western blotting.

PEG-A1 treatment in acute retinal and brain injury mouse models: A pharmaceutical grade of PEG-A1 was provided as a kind gift from Bio-Cancer Treatment International Limited (BCT, Hong Kong). PEG-A1 is a recombinant human arginase (rhArg) covalently attached to methoxy polyethylene glycol (mPEG-SPA; MW 5,000) via succinamide propionic acid (SPA) linker to increase its stability and half-life in vivo (half-life=3 days vs a few minutes for the native enzyme). Methoxy polyethylene glycol propionic acid (MW 5,000—Sigma, Catalog number 88908-1G-F) was used as vehicle control. Animals were injected intraperitoneally with a dose of 25 mg/kg of PEG-A1 or PEG without arginase (vehicle control) at 3 hours after brain or retina injury and at day 4 for mice sacrificed at day 7. The dose was selected based on preliminary dose response studies and dosing interval was based on the PEG-A1 in vivo half-life of 3 days.

The stroke experiment involved behavioral scoring and manual quantification of the infarct size, and was conducted in a blinded fashion following the STAIR criteria. The surgeon performing the surgery, behavioral analysis and infarct size analysis was provided with drug treatment (PEG-A1) and control vehicle (PEG) that are masked and coded. Mice were randomly assigned to the coded treatments and unmasking was performed after completion of experiment and analysis. Another cohort of mice was subjected to MCAO without treatment to compare its outcomes to the treatment groups.

Infarct size analysis: Brain infarct size was determined based on TTC staining of brain sections as described in Eldahshan, et al., American Journal of Physiology Heart and Circulatory Physiology, 316:H1192-h201 (2019). Images of TTC-stained sections were analyzed in a blinded fashion using ImageJ software [National Institutes of Health (NIH)], and the infarct size was calculated with edema correction using the following formula: 100×[nonstroked−(stroked−infarct)]/nonstroked.

Sensorimotor assessment: Bederson score was conducted as described in Bieber, et al., Stroke, 50:2875-82 (2019). Briefly, Mice were scored based on the following five parameters: 0) no observable deficit; 1) forelimb flexion; 2) forelimb flexion and decreased resistance to lateral push; 3) circling; 4) circling and spinning around the cranial-caudal axis; and 5) no spontaneous movement.

Results:

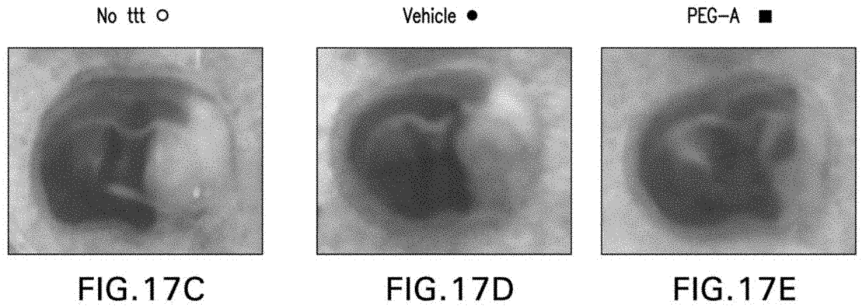

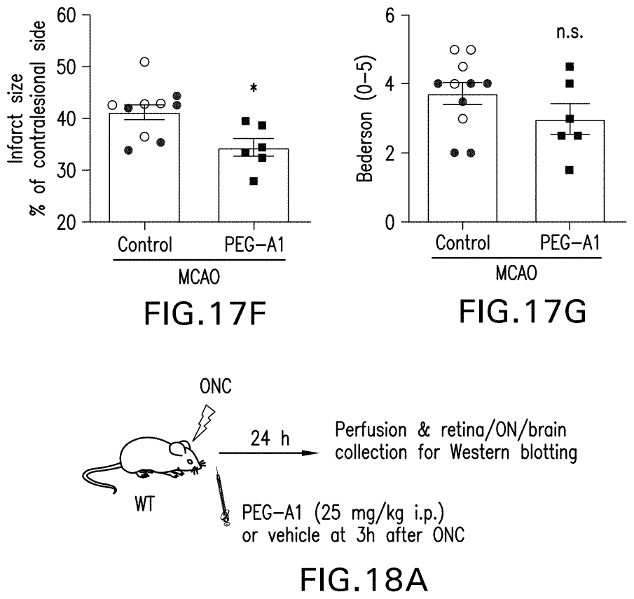

To evaluate the penetration of PEG-A1 into the brain and the effect on outcomes after stroke, WT mice were subjected to 60 minutes MCAO and treated with PEG-A1 or vehicle (PEG only) at reperfusion. Mice were sacrificed and trans-cardially perfused with PBS at 24 hours after stroke (FIG. 17A). Western blotting analysis showed a strong band of anti-PEG immunoreactivity in the stroked brains, which was also detectable with an anti-A1 antibody, thus confirming drug penetration to brain tissue after stroke (FIG. 17B). To test the neuroprotective actions of PEG-A1 in ischemic stroke, brain sections were stained with triphenyl tetrazolium chloride (TTC) for infarct analysis. Mice were also tested for behavioral deficit using Bederson score before sacrifice at 24 hours. PEG-A1 treated mice showed significant reduction in infarct size (34 vs 41%) compared to vehicle treatment (FIG. 17C-17E, 17F). Furthermore, PEG-A1 treated mice showed a trend towards improved Bederson score that did not reach statistical significance (FIG. 17G).

### Example 8. Systemic PEG-A1 Treatment Crosses the Blood-Brain Barrier and Provides Neuroprotection after ONC

Results:

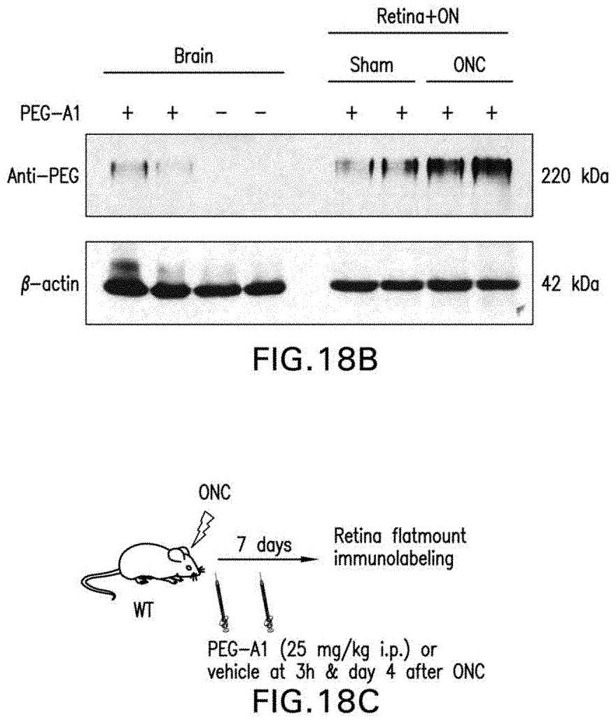

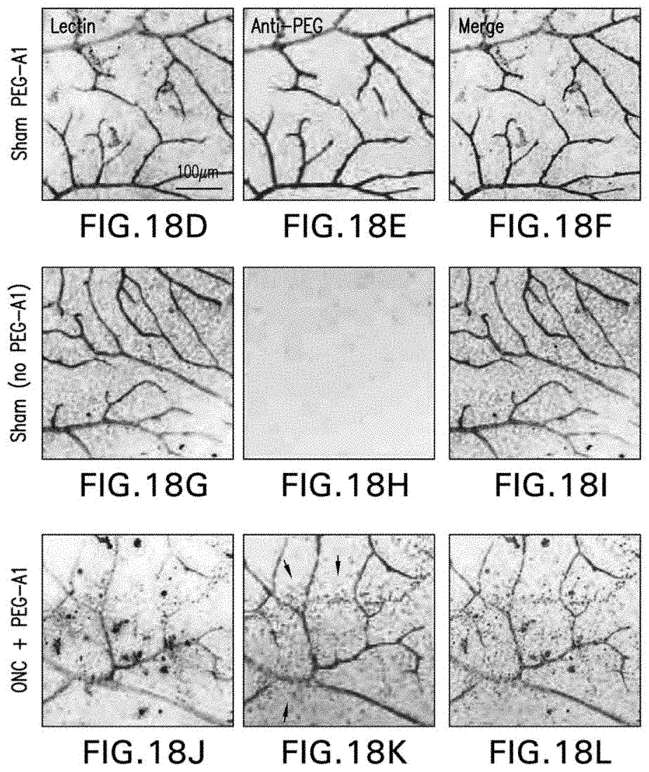

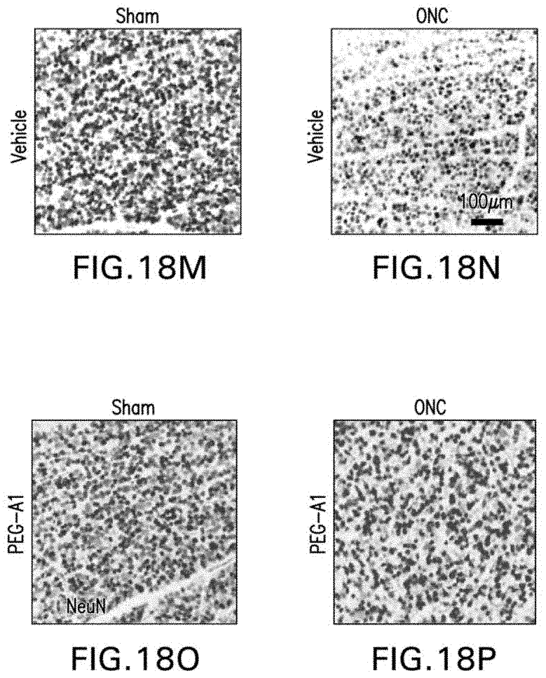

The effects of systemic PEG-A1 delivery were tested in traumatic optic neuropathy (TON) using the optic nerve crush (ONC) model. Mice were subjected to ONC and treated with PEG-A1 three hours later. At 24 hours later the mice were sacrificed and perfused (FIG. 18A). Another group was given a booster dose at day 4 and then sacrificed without perfusion at day 7 (FIG. 18C). PEG-A1 showed effective penetration into the retina/optic nerve tissue homogenates but not into the brain or sham contralateral retina/optic nerve at 24 hours after ONC as measured by western blotting (FIG. 18B) and flat-mount immunolabeling at 7 days (FIGS. 18D-18L). Retina flat-mounts collected at 7 days post-ONC showed significant neuroprotection (78 vs 62% in the vehicle group) as measured by NeuN labeling and quantification (FIG. 18M-18P, 18Q)

### Example 9. PEG-A1 Provides Neuroprotection Ex Vivo

Materials and Methods:

Ex vivo explants: Retinal explants were prepared according to published protocols with modifications (Alarautalahti, et al., Investigative Ophthalmology & Visual Science, 60:1914-27 (2019); Johnson, et al., Investigative Ophthalmology & Visual Science, 49:3503-12 (2008)). Mice were deeply anesthetized by ketamine/xylazine mixture then killed by cervical dislocation. Retinal eye cups were gently dissected and immersed in ice-cold HBSS containing penicillin (100 U/mL) and streptomycin (100 μg/mL). Under a dissecting microscope, the retinas were gently removed and cut radially into four separate equal-sized petals. The retina petals were placed in cell culture inserts (12 mm diameter, 0.4 μm pore, Millipore) with the inner retina facing up. The inserts were placed in 24-well plate in Neurobasal A medium supplemented with 2% B27 (Invitrogen), 1% N2 (Invitrogen), 2 mM GlutaMAX (Invitrogen), penicillin (100 U/mL), and streptomycin (100 μg/mL). Plates were placed in a humidified 37° C. incubator with 5% CO2. For OGD/R experiments, explants were placed in DMEM no glucose medium in a hypoxia chamber (ProOx 110, Biospherix, <1% O2, 94% N2, and 5% CO2 at 37° C.) for the desired duration (1, 3, or 5 hours) then switched back to Neurobasal A growth medium and reoxygenated in a regular incubator (95% air, 5% CO2 at 37° C.) for the reminder of a total 24-hour experiment (23, 21, or 19 hours respectively) to model the ischemia-reperfusion injury. At the end of the experiment, explants were fixed in 4% PFA and processed for flat-mount staining.

One experiment was conducted on explants isolated from myeloid A1 knock out (M-A1 KO) mice and foxed (A1^{f/f}) controls. The breeding and characterization of these mice were described in Fouda, A Y, et al., Cell death & Disease, 9:1001 (2018).

Oxygen-glucose deprivation/reoxygenation (OGD/R) protocol: Oxygen-glucose deprivation/reoxygenation (OGD/R) in vitro was used to mimic in vivo ischemia/reperfusion injury. To achieve OGD, neuron culture was incubated in a hypoxia chamber (ProOx 110, Biospherix, <1% O2, 94% N2, and 5% CO2 at 37° C.) and glucose free DMEM medium and in some experiments this was followed by “reoxygenation” in complete medium under normoxic conditions (95% air, 5% CO2 at 37° C.). Specific durations of OGD or OGD/R were as follows: Differentiated R28 cells were subjected to OGD for 3 or 24 hours and treated with PEG-A1 (1 μg/ml) for the duration of experiment then LDH release assay (Roche) was conducted on the supernatant following the manufacturer's instructions. Another passage of R28 cells were exposed to 3 hours of OGD followed by 3 hours of reoxygenation and treated with PEG-A1 (0.1 and 1 μg/ml) at reoxygenation followed by MTT (3-[4,5-dimethylthiazol-2-yl]-2,5 diphenyl tetrazolium bromide) assay as described in Jittiporn, et al., Microvasc Res, 93:72-9 (2014).

Primary retinal neurons were subjected to OGD for 6 hours followed by reoxygenation for 18 hours and treated with PEG-A1 (1 μg/ml) or the arginase inhibitor ABH (2(S)-amino-6-boronohexanoic acid, 100 μM) at reoxygenation followed by LDH release assay.

Results:

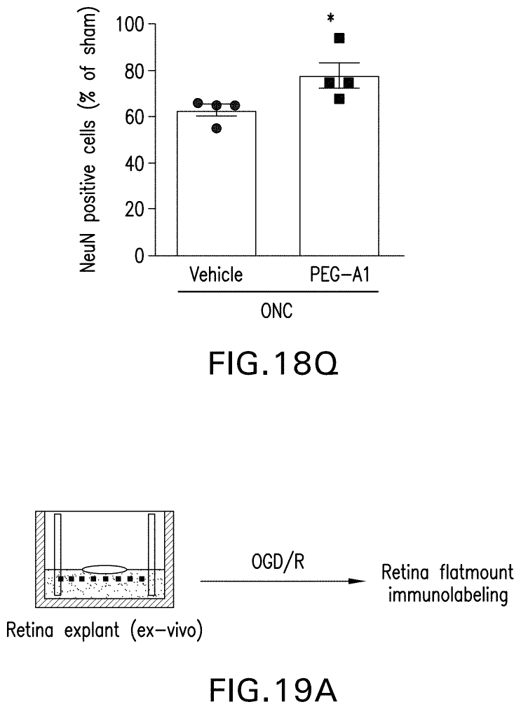

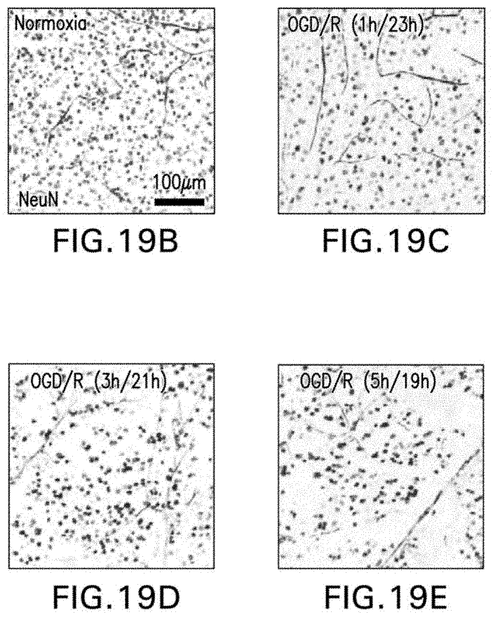

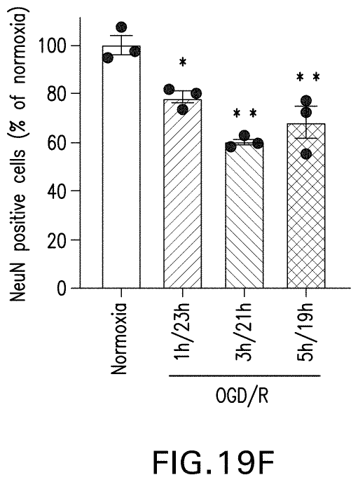

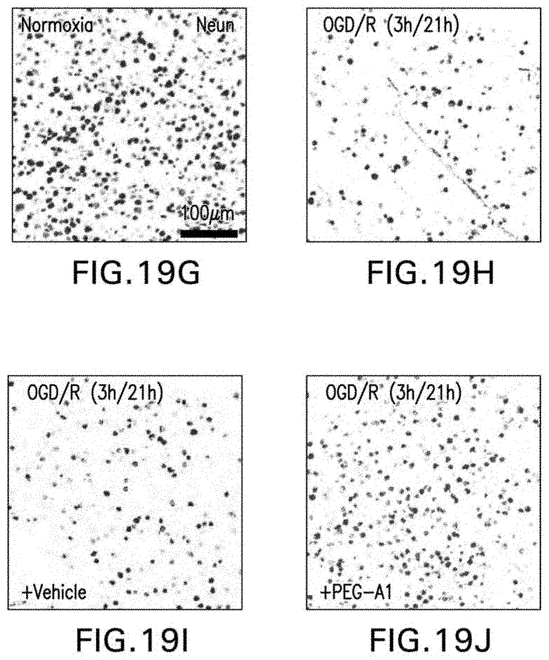

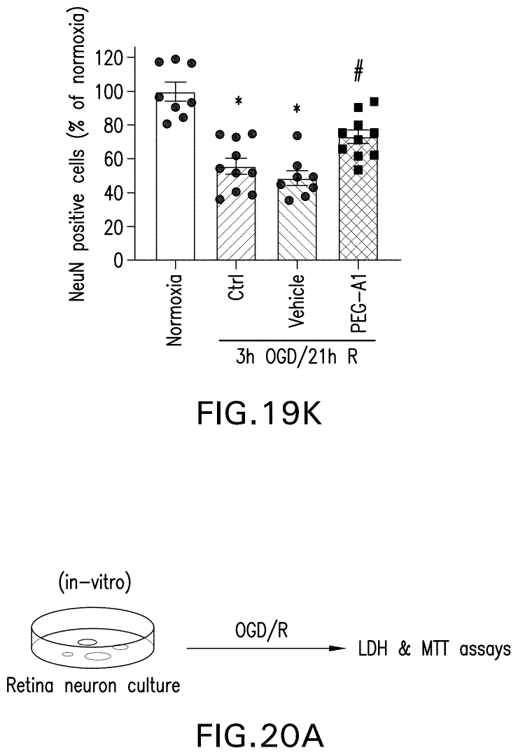

After establishing the systemic protective role of PEG-A1 treatment, the direct effect of PEG-A1 on retinal neuronal survival ex vivo was examined. The retinal IR model was mimicked by subjecting retinal explants to oxygen-glucose deprivation/reperfusion (OGD/R) ex vivo (FIG. 19A). An OGD time-response experiment established 3 hours of OGD/21 hours of reoxygenation to be optimal for studying retinal neurodegeneration ex vivo with about 60% neuronal survival as measured by NeuN flat-mount staining (FIG. 19B-19E, 19F). To test the effect of A1 treatment, retina explants were subjected to 3 hours of OGD then treated at reoxygenation with PEG-A1 or control PEG. A1 treated explants showed significant neuronal preservation (73% survival) as compared to untreated or PEG only treated explants which showed 55 and 48% survival respectively (FIG. 19G-19J, 19K).

### Example 10. PEG-A1 is not Neuroprotective In Vitro

Materials and Methods:

R28 retinal neuronal-like cells: Cells were purchased from Kerafast, Inc. Boston, Mass. 02210. Cells were cultured and differentiated as described Kong, et al., Exp Eye Res., 151:82-95 (2016) and McLaughlin, et al., Frontiers in Aging Neuroscience, 10:267 (2018). Cell passages 67 to 70 were used for experiments. R28 cells were cultured in DMEM with low glucose (Sigma) supplemented with 10% FBS and 1% Penicillin-streptomycin, 1% Amphotericin B. For differentiation, cells were passaged into Laminin coated plates and supplemented with 250 μM pCPT-cAMP for overnight incubation.

Primary retinal neurons: Primary retinal mixed neurons were isolated from newborn Sprague-Dawley (SD) rat pups at postnatal day (P) 1-2 as described in Peŕigolo-Vicente, et al., Biochem Biophys Res Commun., 449:477-82 (2014) and Han, et al., Investigative Ophthalmology & Visual Science, 55:5616-28 (2014) with modifications. Retinas from ten pups were collected and washed twice with ice-cold 1×PBS. They were digested with 0.5% trypsin for 3-4 min at 37° C., followed by trypsin inactivation with the culture medium, DMEM/F12 (Gibco, Grand Island, N.Y.) plus 10% FBS and 1% Penicillin-Streptomycin (P/S) solution (Invitrogen, Grand Island, N.Y.). The retina tissue was mechanically dissociated by pipetting several times until cells were dispersed. Cells were then filtered through a 40 μm nylon-mesh filter, collected by centrifugation at 1000 rpm for 10 min, re-suspended in culture medium, and plated in 6 or 12 well-cell culture plates (Corning, Corning, N.Y.) coated with Poly-D-lysine hydrobromide (PDL, Sigma) at a density of 0.5×106 cells/mL. The plates were maintained in a humidified CO₂ incubator at 37° C. and 5% CO₂. At days 1, 2, and 3 in vitro (DIV), half of the medium was aspirated and replaced with Neurobasal A medium containing 1×B-27 (GIBCO), 1× glutaMAX (GIBCO) and 1% P/S. Experiments were conducted at DIV 6. Immunofluorescence studies confirmed the high purity of cultured neurons using the neuron marker; Neuron-specific class III beta-tubulin (TuJ1), and glia marker; Glial fibrillary acidic protein (GFAP).

Results:

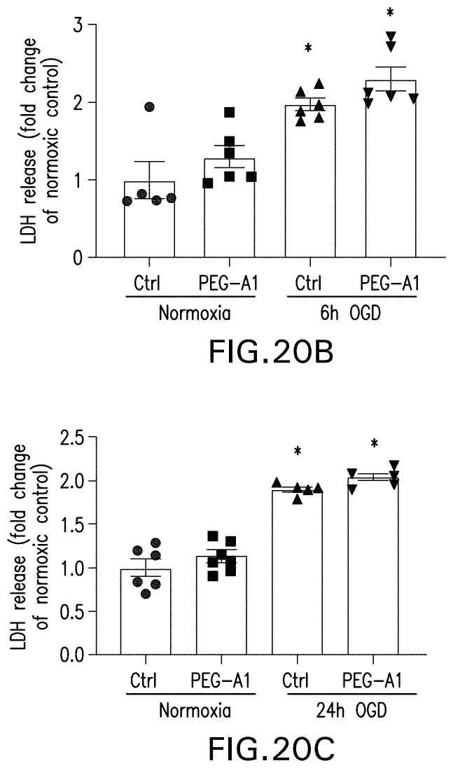

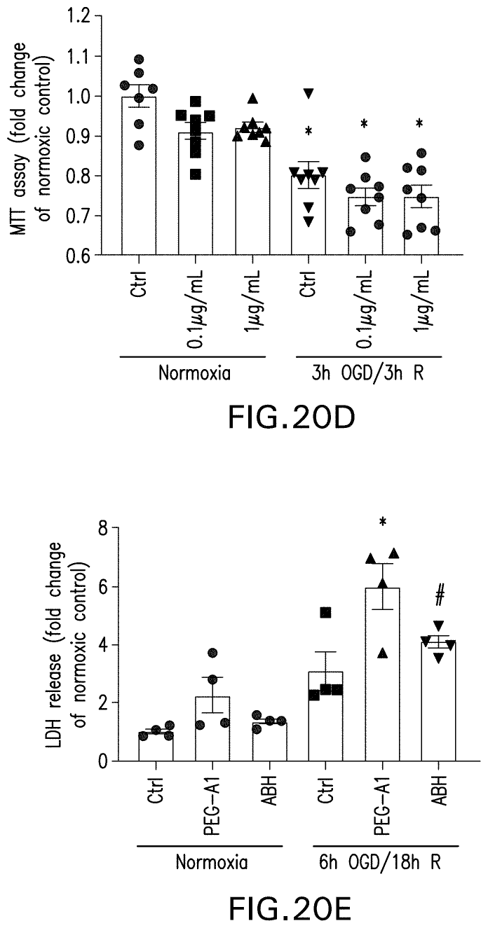

To test the direct effect of PEG-A1 treatment in vitro, retina neuron cultures were subjected to different durations of OGD (FIG. 20A). R28 cells subjected to 6 or 24 hours of OGD and treated with PEG-A1 (1 μg/mL) throughout the duration of experiment did not show any differences in cell death with or without treatment as measured by LDH release assay (FIG. 20B, 20C). Furthermore, R28 cells subjected to 3 hours of OGD then 3 hours of reoxygenation showed a 20% reduction in cell viability but there was no difference with or without PEG-A1 (0.1 and 1 μg/mL) treatment as measured by MTT assay (FIG. 20D). Finally, primary rat retinal mixed neurons subjected to 6 hours of OGD and 18 hours of reperfusion showed increased cell death with PEG-A1 treatment as measured by LDH release. Similarly, treatment with the arginase inhibitor, ABH (100 lead to a trend towards increased cell death, yet it did not reach statistical significance (FIG. 20E).

### Example 11. Retina Explants from Myeloid A1 KO Mice Show Similar Neurodegeneration to Control Mice Subjected to Oxygen-Glucose Deprivation Reoxygenation (OGD/R) Insult

Materials and Methods:

BMDMs: Bone marrow-derived macrophages (BMDMs) were freshly isolated from WT mice and differentiated in culture as described in Fouda, A Y, et al., Cell death & Disease, 9:1001 (2018). To determine PEG-A1 intracellular uptake by macrophages, BMDMs were treated overnight with PEG-A1 or vehicle and then washed several times with phosphate buffered saline (PBS) before collection in RIPA buffer for Western blotting.

Results:

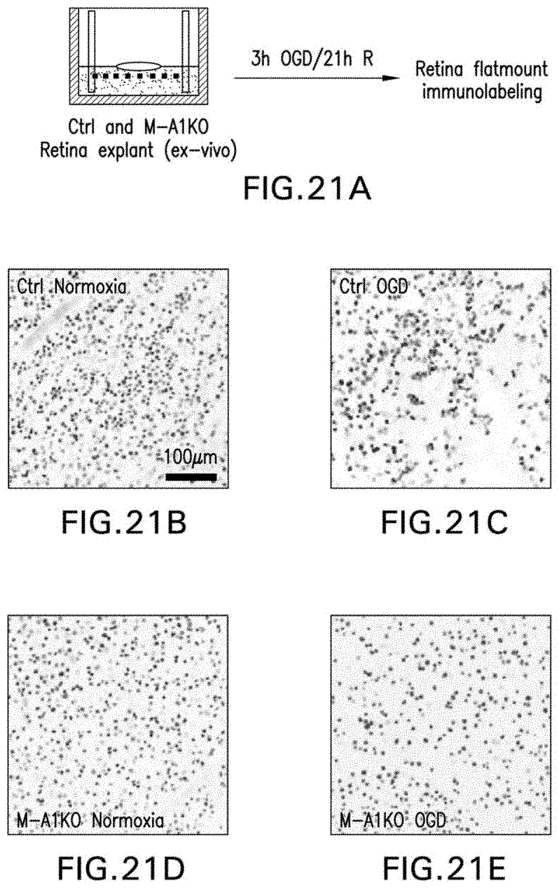

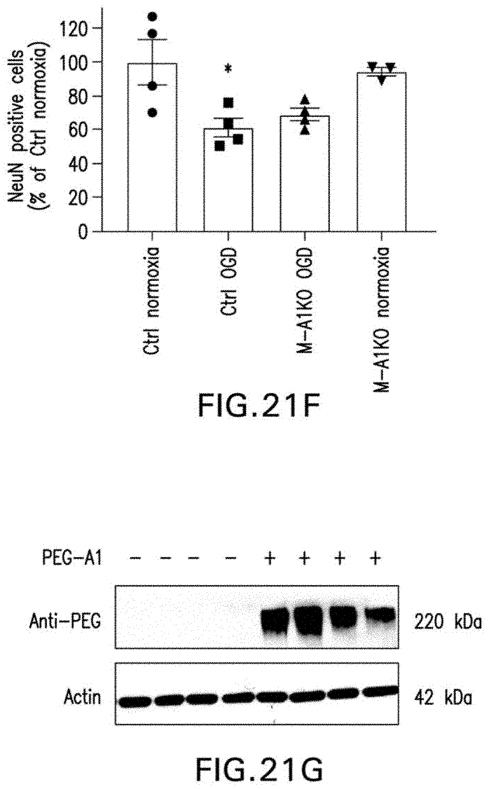

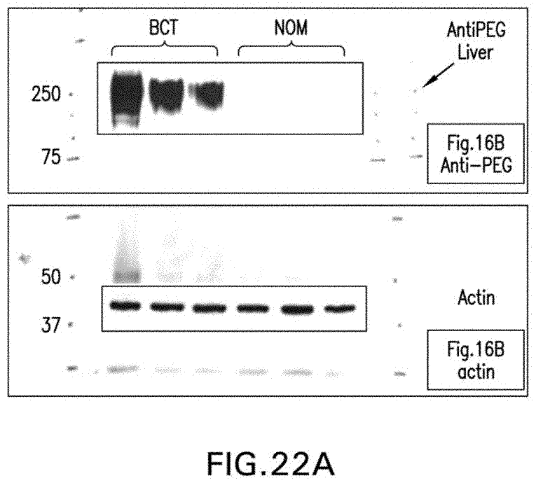

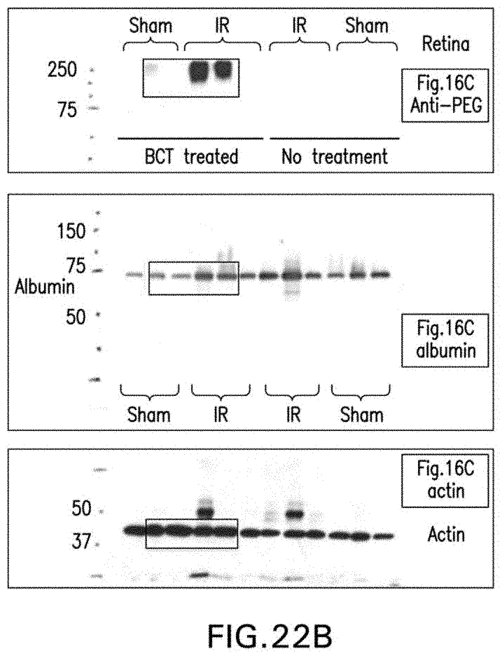

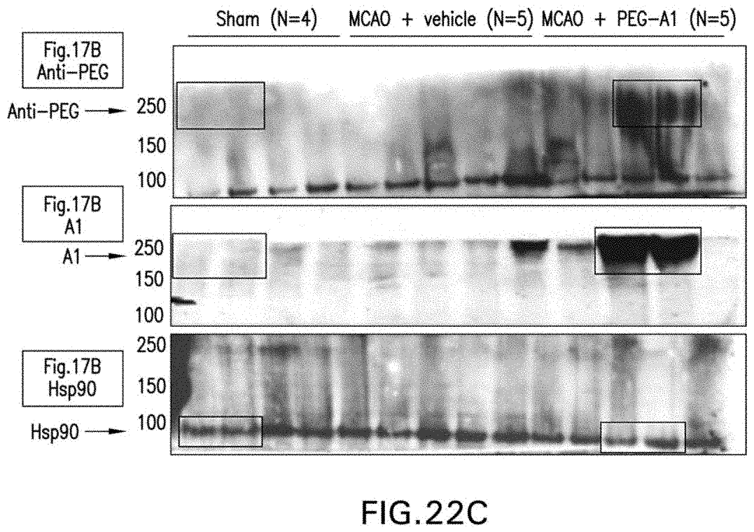

Previous studies have shown that myeloid specific A1 deletion using the LysM cre mice leads to increased neurodegeneration in the retinal IR model (Fouda, A Y, et al., Cell death & Disease, 9:1001 (2018)). However, characterization of the LysM cre promoter showed a 10% neuronal recombination in the retina (Fouda, A Y, et al., Investigative Ophthalmology & Visual Science, 61:51 (2020)). To test the effect of possible neuronal A1 deletion on the previously reported IR outcome in these mice, control and myeloid-specific A1 KO retina explants were subjected to OGD/R (3 hours/21 hours) (FIG. 21A). The explant model excludes any effects due to infiltrating myeloid cells. Neurodegeneration was not different between the two groups suggesting that the in vivo phenotype after IR is primarily due to lack of A1 in infiltrating myeloid cells (FIG. 21B-21E, 21F). To further test if macrophage can incorporate PEG-A1, BMDMs were treated with PEG-A1 or vehicle overnight and then washed with PBS several times before Western blotting. Macrophage treated with PEG-A1 and analyzed by western blotting using anti-PEG antibody showed a strong band in cell lysate, thus suggesting intracellular uptake of PEG-A1 had occurred (FIG. 21G).

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method of inducing or promoting the regression of retinal neovascularization induced injury in a subject in need thereof, comprising administering to the eye of the subject a composition comprising recombinant arginase 1 in an amount effective to promote reparative angiogenesis and decrease retinal neovascularization in the eye to induce or promote the regression of retinal neovasculariation.

2. The method of claim 1, wherein the recombinant arginase 1 is PEGylated.

3. The method of claim 1, wherein the recombinant arginase 1 is administered to the eye of the subject by intravitreal injection.

4. The method of claim 1, wherein the recombinant arginase 1 is administered to the eye of the subject by topical application.

5. The method of claim 1, wherein the subject in need thereof has retinopathy of prematurity (ROP), diabetic retinopathy, age-related macular degeneration, or retinal vein occlusion.

6. The method of claim 1, wherein reparative angiogenesis is promoted by an increase in the levels of growth factors in the retina.

7. The method of claim 6, wherein the growth factors are FGF2 and CNTF.

8. The method of claim 1, wherein the regression of retinal neovascularization is induced by decreased the production and expression of inflammatory molecules in the retina.

9. The method of claim 8, wherein the inflammatory molecules are TNFα, IL-6, and MCP1.

10. A method of reducing or inhibiting the progression of retinal neovascularization related disease in a subject in need thereof, comprising administering to the subject a composition comprising recombinant arginase 1 in an amount effective to promote reparative angiogenesis and decrease retinal neovascularization in the eye.

11. The method of claim 10, wherein the recombinant arginase 1 is PEGylated.

12. The method of claim 10, wherein the recombinant arginase 1 is administered to the eye of the subject by intravitreal injection.

13. The method of claim 10, wherein the recombinant arginase 1 is administered to the eye of the subject by topical application.

14. The method of claim 10, wherein the retinal neovascularization related disease is retinopathy of prematurity (ROP), diabetic retinopathy, age-related macular degeneration, or retinal vein occlusion.

15. A method of treating retinopathy of prematurity in an infant in need thereof, comprising administering to the infant a composition comprising recombinant arginase 1 in an amount effective to promote reparative angiogenesis and decrease retinal neovascularization in the eye to induce or promote the regression of retinal neovasculariation.

16. The method of claim 15, wherein the recombinant arginase 1 is PEGylated.

17. The method of claim 15, wherein the recombinant arginase 1 is administered to the eye of the infant by intravitreal injection.

18. The method of claim 15, wherein the recombinant arginase 1 is administered to the eye of the infant by topical application.

19. The method of claim 15, wherein composition is administered to the infant once a week.

20. The method of claim 15, wherein the composition is administered to the infant once a month.

21. The method of claim 15, wherein administration of recombinant arginase prevents blindness in the infant.

22. An ophthalmic composition comprising pegylated arginase 1.

23. A method of preventing or treating neurodegeneration in a subject in need thereof, comprising administering to the subject a composition comprising recombinant arginase 1 in an amount effective to promote neuroprotection or neurogenesis.

24. The method of claim 23, wherein the recombinant arginase 1 is PEGylated.

25. The method of claim 23, wherein neurodegeneration in a subject is the result of an ischemic injury to the brain or eye.

26. The method of claim 25, wherein the ischemic injury is an ischemic stroke or ischemic retinopathies.

27. The method of claim 25, wherein systemic administration of recombinant arginase 1 provides promotes neuroprotection from ischemic injury to the retina or brain by penetrating the blood-retina or blood brain barrier of a subject with prior ischemic injury to the retina or brain.

28. The method of claim 23, wherein neurodegeneration in a subject is the result of acute retinal injury, wherein the acute retinal injury results in traumatic optic neuropathy.

29. The method of claim 28, wherein systemic administration of recombinant arginase 1 provides promotes neuroprotection from traumatic optic neuropathy by penetrating the blood-retina barrier of a subject with acute retinal injury.


SEQUENCE LISTING (3 sequences)

SEQ ID NO: 1 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 2 - protein, 330 aa, Homo sapiens
MSAKSRTIGIIGAPFSKGQPRGGVEEGPTVLRKAGLLEKLKEQVTQNFLILECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 3 - protein, 236 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVTRTVNTAVAITLACFGLAREGNHKPIDYLNPPK